(19) United States
(12) Patent Application Publication
(10) Pub. No.: US 2022/0257260 A1
(43) Pub. Date: Aug. 18, 2022

(54) FILAMENTARY DEVICES FOR TREATMENT OF VASCULAR DEFECTS

(71) Applicant: MICROVENTION, INC., Aliso Viejo, CA (US)

(72) Inventors: Todd Hewitt, Laguna Niguel, CA (US); Joseph Emery, Riverside, CA (US); Hung Tran, Midway City, CA (US); John Vu, Santa Ana, CA (US); Karishma Desai, Irvine, CA (US); Parker Milhous, Santa Ana, CA (US)

(21) Appl. No.: 17/550,085

(22) Filed: Dec. 14, 2021

Related U.S. Application Data
(60) Provisional application No. 63/127,517, filed on Dec. 18, 2020.

(51) Int. Cl. A61B 17/12

(52) U.S. Cl. CPC A61B 17/12172; A61B 17/12113

(57) ABSTRACT

Devices and methods for treatment of a patient's vasculature are described. Embodiments may include an implant having multiple mesh layers. A first and second layer of the implant may be formed by folding over a single mesh and the third layer may be formed from a different mesh. The first, second, and third layers may be either a stasis or a structural layer and may be arranged in different orders. Embodiments may also include structures to assist in the expansion of the device. These structures may include an expandable loop or reinforcing finger extensions. Embodiments may also include a corrugation or ridge around a middle section of the implant to assist in deployment.

### CROSS-REFERENCE TO RELATED APPLICATIONS

This application claims the benefit of priority under 35 U.S.C. § 119(e) from U.S. Provisional Application Ser. No. 63/127,517, filed Dec. 18, 2020, which is hereby incorporated by reference in its entirety for all purposes.

### STATEMENT REGARDING FEDERALLY SPONSORED RESEARCH OR DEVELOPMENT

Not applicable.

### FIELD OF THE INVENTION

Embodiments of devices and methods herein are directed to blocking a flow of fluid through a tubular vessel or into a small interior chamber of a saccular cavity or vascular defect within a mammalian body. More specifically, embodiments herein are directed to devices and methods for treatment of a vascular defect of a patient including some embodiments directed specifically to the treatment of cerebral aneurysms of patients.

### BACKGROUND

The mammalian circulatory system is comprised of a heart, which acts as a pump, and a system of blood vessels which transport the blood to various points in the body. Due to the force exerted by the flowing blood on the blood vessel the blood vessels may develop a variety of vascular defects. One common vascular defect known as an aneurysm results from the abnormal widening of the blood vessel. Typically, vascular aneurysms are formed as a result of the weakening of the wall of a blood vessel and subsequent ballooning and expansion of the vessel wall. If, for example, an aneurysm is present within an artery of the brain, and the aneurysm should burst with resulting cranial hemorrhaging, death could occur.

Surgical techniques for the treatment of cerebral aneurysms typically involve a craniotomy requiring creation of an opening in the skull of the patient through which the surgeon can insert instruments to operate directly on the patient's brain. For some surgical approaches, the brain must be retracted to expose the parent blood vessel from which the aneurysm arises. Once access to the aneurysm is gained, the surgeon places a clip across the neck of the aneurysm thereby preventing arterial blood from entering the aneurysm. Upon correct placement of the clip the aneurysm will be obliterated in a matter of minutes. Surgical techniques may be effective treatment for many aneurysms. Unfortunately, surgical techniques for treating these types of conditions include major invasive surgical procedures which often require extended periods of time under anesthesia involving high risk to the patient. Such procedures thus require that the patient be in generally good physical condition in order to be a candidate for such procedures.

Various alternative and less invasive procedures have been used to treat cerebral aneurysms without resorting to major surgery. One approach to treating aneurysms without the need for invasive surgery involves the placement of sleeves or stents into the vessel and across the region where the aneurysm occurs. Such devices maintain blood flow through the vessel while reducing blood pressure applied to the interior of the aneurysm. Certain types of stents are expanded to the proper size by inflating a balloon catheter, referred to as balloon expandable stents, while other stents are designed to elastically expand in a self-expanding manner. Some stents are covered typically with a sleeve of polymeric material called a graft to form a stent-graft. Stents and stent-grafts are generally delivered to a preselected position adjacent a vascular defect through a delivery catheter. In the treatment of cerebral aneurysms, covered stents or stent-grafts have seen very limited use due to the likelihood of inadvertent occlusion of small perforator vessels that may be near the vascular defect being treated.

In addition, current uncovered stents are generally not sufficient as a stand-alone treatment. In order for stents to fit through the microcatheters used in small cerebral blood vessels, their density is usually reduced such that when expanded there is only a small amount of stent structure bridging the aneurysm neck. Thus, they do not block enough flow to cause clotting of the blood in the aneurysm and are thus generally used in combination with vaso-occlusive devices, such as the coils discussed above, to achieve aneurysm occlusion.

Some procedures involve the delivery of embolic or filling materials into an aneurysm. The delivery of such vaso-occlusion devices or materials may be used to promote hemostasis or fill an aneurysm cavity entirely. Vaso-occlusion devices may be placed within the vasculature of the human body, typically via a catheter, either to block the flow of blood through a vessel with an aneurysm through the formation of an embolus or to form such an embolus within an aneurysm stemming from the vessel. A variety of implantable, coil-type vaso-occlusion devices are known. The coils of such devices may themselves be formed into a secondary coil shape, or any of a variety of more complex secondary shapes. Vaso-occlusive coils are commonly used to treat cerebral aneurysms but suffer from several limitations including poor packing density, compaction due to hydrodynamic pressure from blood flow, poor stability in wide-necked aneurysms, and complexity and difficulty in the deployment thereof as most aneurysm treatments with this approach require the deployment of multiple coils. Coiling is less effective at treating certain physiological conditions, such as wide neck cavities (e.g. wide neck aneurysms) because there is a greater risk of the coils migrating out of the treatment site.

A number of aneurysm neck bridging devices with defect spanning portions or regions have been attempted, however, none of these devices have had a significant measure of clinical success or usage. A major limitation in their adoption and clinical usefulness is the inability to position the defect spanning portion to assure coverage of the neck. Existing stent delivery systems that are neurovascular compatible (i.e. deliverable through a microcatheter and highly flexible) do not have the necessary rotational positioning capability. Another limitation of many aneurysm bridging devices described in the prior art is the poor flexibility. Cerebral blood vessels are tortuous, and a high degree of flexibility is required for effective delivery to most aneurysm locations in the brain.

What has been needed are devices and methods for delivery and use in small and tortuous blood vessels that can substantially block the flow of blood into an aneurysm, such as a cerebral aneurysm, with a decreased risk of inadvertent aneurysm rupture or blood vessel wall damage. In addition, what has been needed are methods and devices suitable for blocking blood flow in cerebral aneurysms over an extended period of time without a significant risk of deformation, compaction or dislocation.

Intrasaccular occlusive devices are part of a newer type of occlusion device used to treat various intravascular conditions including aneurysms. They are often more effective at treating these wide neck conditions, or larger treatment areas. The intrasaccular devices comprise a structure which sits within the aneurysm and provides an occlusive effect at the neck of the aneurysm to help limit blood flow into the aneurysm. The rest of the device comprises a relatively conformable structure that sits within the aneurysm helping to occlude all or a portion of the aneurysm. Intrasaccular devices typically conform to the shape of the treatment site. These devices also occlude the cross section of the neck of the treatment site/aneurysm, thereby promoting clotting and causing thrombosis and closing of the aneurysm over time. In larger aneurysms, there is a risk of compaction where the intrasaccular device can migrate into the aneurysm and leave the neck region.

Occlusive devices are used for a variety of reasons within the vascular system, such as to limit blood flow to aneurysms to reduce risk of rupture. Other reasons include vessel shutdown and occluding various structures such as arteriovenous malformations, fistula, left atrial appendage, fallopian tubes. One class of occlusive devices are known as intrasaccular devices and are used in treating aneurysms. Aneurysms are typically occluded with small embolic coils that fill the space of the aneurysm, typically a number of coils are used in the procedure to sufficiently occlude or fill the target space. The procedure typically also includes a balloon or stent placed adjacent the aneurysm location to reduce the risk of coils falling out of the aneurysm and migrating elsewhere. Even with these additional devices in use, there is still a risk of the coils sticking out of the aneurysm.

Occlusive devices should have certain characteristics. First, the implants should be deliverable with low force through small microcatheters (for neurovascular applications, “17 size” catheters are the most optimal). Second, the implants should have high enough radial forces to fully deploy and remain positioned within the aneurysm or parent artery after detachment (not migrate). Third, the implants should have high metal surface area to promote flow stasis, which is a reduction of blood flow so as to promote stagnation and clotting.

The following embodiments describe multiple layer implants that achieve the desired characteristics.

### SUMMARY

An occlusion device is described that is used to treat a variety of conditions, including aneurysms and neurovascular aneurysms, in particular, bifurcation aneurysms. In some embodiments, the occlusion device is configured as an intrasaccular device.

The presented embodiments shall generally relate to occlusive devices that include multiple layers and achieve the desired characteristics of being deliverable with low force through small microcatheters, having a high enough radial force to fully deploy and remain positioned within the aneurysm after detachment, and having a high metal surface area for flow stasis.

In many embodiments, a device for treatment of a patient's cerebral aneurysm is described. The device includes a permeable shell comprising first, second, and third layers, a proximal end, a distal end, wherein the permeable shell has a radially constrained elongated state configured for delivery within a catheter lumen and an expanded state with a longitudinally shortened configuration relative to the radially constrained state, wherein the first and second layers are formed from a first plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell, and wherein the third layer is formed from a second plurality of elongate filaments that are woven together to form a mesh, wherein a first end of each of the second plurality of elongate filaments are gathered at the proximal end of the permeable shell.

In many embodiments, a method for treating a cerebral aneurysm having an interior cavity and a neck is described. The method includes the steps of advancing an implant in a microcatheter to a region of interest in a cerebral artery, wherein the implant comprises: a permeable shell comprising first, second, and third layers, a proximal end, a distal end, wherein the permeable shell has a radially constrained elongated state configured for delivery within a catheter lumen and an expanded state with a longitudinally shortened configuration relative to the radially constrained state, wherein the first and second layers are formed from a first plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell, and wherein the third layer is formed from a second plurality of elongate filaments that are woven together to form a mesh, wherein a first end of each of the second plurality of elongate filaments are gathered at the proximal end of the permeable shell; deploying the implant within the cerebral aneurysm, wherein the permeable shell expands to the expanded state in the interior cavity of the aneurysm; and withdrawing the microcatheter from the region of interest after deploying the implant.

In some embodiments, the mesh formed by the first plurality of elongate filaments is inverted in the middle portion to form the first and second layers.

In some embodiments, a second end of each of the second plurality of elongate filaments is gathered at the distal end of the permeable shell.

In some embodiments, each of the second plurality of elongate filaments forms a loop at or near a distal end of the permeable shell. In some embodiments, each of the first and second ends of the second plurality of elongate filaments is gathered at the proximal end of the permeable shell.

In some embodiments, the third layer is located between the first and second layers.

In some embodiments, the third layer is an innermost surface of the permeable shell.

In some embodiments, the third layer is an outermost surface of the permeable shell.

In some embodiments, the third layer is a stasis layer. In some embodiments, the first and second layers are structural layers.

In some embodiments, the first and second layers are stasis layers. In some embodiments, the third layer is a structural layer.

In some embodiments, each of the first plurality of filaments has a diameter between about 0.001″ to about 0.004″. In some embodiments, each of the second plurality of filaments has a diameter between about 0.00025″ to about 0.00075″.

In some embodiments, each of the second plurality of filaments has a diameter between about 0.001″ to about 0.004″. In some embodiments, each of the first plurality of filaments has a diameter between about 0.00025″ to about 0.00075″.

In some embodiments, an average diameter of the first plurality of filaments is larger than an average diameter of the second plurality of filaments.

In some embodiments, an average diameter of the second plurality of filaments is larger than an average diameter of the first plurality of filaments.

In some embodiments, the first plurality of filaments has a larger number of filaments than the second plurality of filaments.

In some embodiments, the second plurality of filaments has a larger number of filaments than the first plurality of filaments.

In some embodiments, the device further includes a marker band located at the proximal end of the permeable shell. In some embodiments, the marker band may gather the proximal and distal ends of the first plurality of elongate filaments and the proximal ends of the second plurality of elongate filaments.

In some embodiments, the distal end of the permeable shell does not include a distal hub.

In some embodiments, the distal end of the permeable shell comprises an opening in communication with an inner cavity of the permeable shell.

In some embodiments, the distal end of each of the second plurality of elongate filaments are not gathered together in a hub. In some embodiments, the distal end of each of the second plurality of elongate filaments are gathered together in a hub.

In some embodiments, an expanded state of the third layer has an open distal end.

In many embodiments, a device for treatment of a patient's cerebral aneurysm is described. The device includes a permeable shell comprising first and second layers, a proximal end, and a distal end, and a radially expanding member located between the first and second layers, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell.

In many embodiments, a method for treating a cerebral aneurysm having an interior cavity and a neck is described. The method includes the steps of advancing an implant in a microcatheter to a region of interest in a cerebral artery, wherein the implant comprises: a permeable shell comprising first and second layers, a proximal end, and a distal end, and a radially expanding member located between the first and second layers, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell; deploying the implant within the cerebral aneurysm, wherein the permeable shell expands to the expanded state in the interior cavity of the aneurysm; and withdrawing the microcatheter from the region of interest after deploying the implant.

In some embodiments, the mesh formed by the plurality of elongate filaments is inverted in the middle portion to form the first and second layers.

In some embodiments, the distal end of the permeable shell does not include a distal hub.

In some embodiments, the distal end of the permeable shell comprises an opening in communication with an inner cavity of the permeable shell.

In some embodiments, the radially expanding member comprises at least one metal filament having a radially constrained elongated state configured for delivery within a catheter lumen and an expanded state, wherein in the expanded state, the at least one metal filament forms a shape comprising a loop. In some embodiments, the loop extends around a center region of the expanded state of the permeable shell.

In some embodiments, the expanded state of the at least one metal filament comprises a loop portion and a non-loop portion, wherein a proximal end of the non-loop portion is gathered at the proximal end of the permeable shell.

In many embodiments, a device for treatment of a patient's cerebral aneurysm is described. The device includes a permeable shell comprising first and second layers, a proximal end, and a distal end, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell; and an expandable frame comprising a core and a plurality of curved supports extending radially from the core, wherein the expandable frame has a radially constrained elongated state configured for delivery within a catheter lumen and an expanded state with a longitudinally shortened configuration relative to the radially constrained state, wherein the plurality of curved supports are located between the first and second layers of the permeable shell.

In many embodiments, a method for treating a cerebral aneurysm having an interior cavity and a neck is described. The method includes the steps of advancing an implant in a microcatheter to a region of interest in a cerebral artery, wherein the implant comprises: a permeable shell comprising first and second layers, a proximal end, and a distal end, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell; and an expandable frame comprising a core and a plurality of curved supports extending radially from the core, wherein the expandable frame has a radially constrained elongated state configured for delivery within a catheter lumen and an expanded state with a longitudinally shortened configuration relative to the radially constrained state, wherein the plurality of curved supports are located between the first and second layers of the permeable shell; deploying the implant within the cerebral aneurysm, wherein the permeable shell expands to the expanded state in the interior cavity of the aneurysm; and withdrawing the microcatheter from the region of interest after deploying the implant.

In some embodiments, the core is a ring.

In some embodiments, in the expanded state, each of the plurality of curved supports follow a contour of the expanded permeable shell.

In some embodiments, the mesh formed by the plurality of elongate filaments is inverted in the middle portion to form the first and second layers.

In some embodiments, the distal end of the permeable shell does not include a distal hub.

In some embodiments, the distal end of the permeable shell comprises an opening in communication with an inner cavity of the permeable shell.

In some embodiments, the plurality of supports comprises between about 3 and about 8 supports.

In some embodiments, the plurality of supports comprises between about 4 and about 6 supports.

In some embodiments, each of the plurality of supports comprise a proximal end and a distal end, wherein each of the plurality of supports extends towards the distal end of the permeable shell, wherein the distal ends of each of the plurality of supports are located in a region between about 40% to about 60% of the total height of expanded state of the permeable shell.

In many embodiments, a device for treatment of a patient's cerebral aneurysm is described. The device includes a resilient self-expanding permeable shell including a radially constrained elongated state configured for delivery within a catheter lumen, an expanded state with a longitudinally shortened configuration relative to the radially constrained state, and a plurality of elongate filaments that are woven together to form a mesh and define a cavity of the permeable shell, the expanded state having a proximal portion, a middle portion, and a distal portion, wherein the middle portion comprises a corrugation that at least partially extends around a perimeter of the permeable shell.

In many embodiments, a method for treating a cerebral aneurysm having an interior cavity and a neck is described. The method includes the steps of advancing an implant in a microcatheter to a region of interest in a cerebral artery, wherein the implant comprises a resilient self-expanding permeable shell including a radially constrained elongated state configured for delivery within a catheter lumen, an expanded state with a longitudinally shortened configuration relative to the radially constrained state, and a plurality of elongate filaments that are woven together to form a mesh and define a cavity of the permeable shell, the expanded state having a proximal portion, a middle portion, and a distal portion, wherein the middle portion comprises a corrugation that at least partially extends around a perimeter of the permeable shell; deploying the implant within the cerebral aneurysm, wherein the permeable shell expands to the expanded state in the interior cavity of the aneurysm; and withdrawing the microcatheter from the region of interest after deploying the implant.

In some embodiments, the permeable shell comprises first and second layers. In some embodiments, the first and second layers are formed from the plurality of elongate filaments that are woven together to form the mesh, wherein each of the plurality of elongate filaments have a first end, a second end, and a middle portion, wherein the first and second ends of each of the plurality of elongate filaments are gathered at the proximal end of the permeable shell, and wherein the middle portion of each of the plurality of elongate filaments is associated with the distal end of the permeable shell. In some embodiments, both of the first and second layers of the permeable shell comprise the corrugation.

In some embodiments, the corrugation defines a plane, and wherein the plane is substantially perpendicular to an axis of the permeable shell extending from the proximal to distal end of the permeable shell.

In some embodiments, the corrugation is a ridge or indentation.

In some embodiments, the mesh formed by the plurality of elongate filaments is inverted in the middle portion to form the first and second layers.

In some embodiments, the distal end of the permeable shell does not include a distal hub.

In some embodiments, the distal end of the permeable shell comprises an opening in communication with an inner cavity of the permeable shell.

In some embodiments, the corrugation extends around the perimeter of the permeable shell.

In some embodiments, the distal end of the permeable shell does not include a distal marker band.

In some embodiments, the distal end of the permeable shell comprises an opening in communication with an inner cavity defined by the permeable shell.

### BRIEF DESCRIPTION OF THE DRAWINGS

FIG. 1 is an elevation view of an embodiment of a device for treatment of a patient's vasculature and a plurality of arrows indicating inward radial force.

FIG. 2 is an elevation view of a beam supported by two simple supports and a plurality of arrows indicating force against the beam.

FIG. 3 is a bottom perspective view of an embodiment of a device for treatment of a patient's vasculature.

FIG. 4 is an elevation view of the device for treatment of a patient's vasculature of FIG. 3.

FIG. 5 is a transverse cross sectional view of the device of FIG. 4 taken along lines 5-5 in FIG. 4.

FIG. 6 shows the device of FIG. 4 in longitudinal section taken along lines 6-6 in FIG. 4.

FIG. 7 is an enlarged view of the woven filament structure taken from the encircled portion 7 shown in FIG. 5.

FIG. 8 is an enlarged view of the woven filament structure taken from the encircled portion 8 shown in FIG. 6.

FIG. 9 is a proximal end view of the device of FIG. 3.

FIG. 10 is a transverse sectional view of a proximal hub portion of the device in FIG. 6 indicated by lines 10-10 in FIG. 6.

FIG. 11 is an elevation view in partial section of a distal end of a delivery catheter with the device for treatment of a patient's vasculature of FIG. 3 disposed therein in a collapsed constrained state.

FIG. 12 illustrates an embodiment of a filament configuration for a device for treatment of a patient's vasculature.

FIGS. 13A-13C depict example embodiments of cross-sections of three-layer occlusion implants.

FIGS. 13D-13G depict various steps during the process for assembling a three-layer implant.

FIG. 13H shows a three-layer flow diverter with a high wire count, small wire stasis layer captured within the “folded over” structural layer.

FIGS. 13I-13K depict example embodiments of cross-sections of three-layer flow diverter implants.

FIG. 14A is an example embodiment of a dual-layer occlusion implant with a reinforcing belt.

FIG. 14B is a horizontal cross-section of the embodiment in FIG. 14A.

FIG. 14C is a vertical cross-section of the embodiment in FIG. 14A.

FIG. 14D is a depiction of a reinforcing belt in an expanded state.

FIG. 15A is an example embodiment of a dual-layer occlusion implant with reinforcing fingers.

FIG. 15B is a horizontal cross-section of the embodiment in FIG. 15A.

FIG. 15C is a vertical cross-section of the embodiment in FIG. 15A.

FIG. 15D is a tube cut to form the reinforcing fingers (pre-shape set).

FIG. 15E is the tube of FIG. 15D in a post-shape set.

FIG. 16A is an example embodiment with a corrugation in a middle region.

FIG. 16B is a vertical cross section of the embodiment of FIG. 16A.

FIG. 17 is a schematic view of a patient being accessed by an introducer sheath, a microcatheter and a device for treatment of a patient's vasculature releasably secured to a distal end of a delivery device or actuator.

FIG. 18 is a sectional view of a terminal aneurysm.

FIG. 19 is a sectional view of an aneurysm.

FIG. 20 is a schematic view in section of an aneurysm showing perpendicular arrows which indicate interior nominal longitudinal and transverse dimensions of the aneurysm.

FIG. 21 is a schematic view in section of the aneurysm of FIG. 20 with a dashed outline of a device for treatment of a patient's vasculature in a relaxed unconstrained state that extends transversely outside of the walls of the aneurysm.

FIG. 22 is a schematic view in section of an outline of a device represented by the dashed line in FIG. 21 in a deployed and partially constrained state within the aneurysm.

FIGS. 23-26 show a deployment sequence of a device for treatment of a patient's vasculature.

FIG. 27 is an elevation view in partial section of an embodiment of a device for treatment of a patient's vasculature deployed within an aneurysm at a tilted angle.

FIG. 28 is an elevation view in partial section of an embodiment of a device for treatment of a patient's vasculature deployed within an irregularly shaped aneurysm.

FIG. 29 shows an elevation view in section of a device for treatment of a patient's vasculature deployed within a vascular defect aneurysm.

### DETAILED DESCRIPTION

Discussed herein are devices and methods for the treatment of vascular defects that are suitable for minimally invasive deployment within a patient's vasculature, and particularly, within the cerebral vasculature of a patient. For such embodiments to be safely and effectively delivered to a desired treatment site and effectively deployed, some device embodiments may be configured for collapse to a low profile constrained state with a transverse dimension suitable for delivery through an inner lumen of a microcatheter and deployment from a distal end thereof. Embodiments of these devices may also maintain a clinically effective configuration with sufficient mechanical integrity once deployed so as to withstand dynamic forces within a patient's vasculature over time that may otherwise result in compaction of a deployed device. It may also be desirable for some device embodiments to acutely occlude a vascular defect of a patient during the course of a procedure in order to provide more immediate feedback regarding success of the treatment to a treating physician.

Intrasaccular occlusive devices that include a permeable shell formed from a woven or braided mesh have been described in US 2017/0095254, US 2016/0249934, US 2016/0367260, US 2016/0249937, and US 2018/0000489, all of which are hereby expressly incorporated by reference in their entirety for all purposes.

Some embodiments are particularly useful for the treatment of cerebral aneurysms by reconstructing a vascular wall so as to wholly or partially isolate a vascular defect from a patient's blood flow. Some embodiments may be configured to be deployed within a vascular defect to facilitate reconstruction, bridging of a vessel wall or both in order to treat the vascular defect. For some of these embodiments, the permeable shell of the device may be configured to anchor or fix the permeable shell in a clinically beneficial position. For some embodiments, the device may be disposed in whole or in part within the vascular defect in order to anchor or fix the device with respect to the vascular structure or defect. The permeable shell may be configured to span an opening, neck or other portion of a vascular defect in order to isolate the vascular defect, or a portion thereof, from the patient's nominal vascular system in order allow the defect to heal or to otherwise minimize the risk of the defect to the patient's health.

For some or all of the embodiments of devices for treatment of a patient's vasculature discussed herein, the permeable shell may be configured to allow some initial perfusion of blood through the permeable shell. The porosity of the permeable shell may be configured to sufficiently isolate the vascular defect so as to promote healing and isolation of the defect, but allow sufficient initial flow through the permeable shell so as to reduce or otherwise minimize the mechanical force exerted on the membrane the dynamic flow of blood or other fluids within the vasculature against the device. For some embodiments of devices for treatment of a patient's vasculature, only a portion of the permeable shell that spans the opening or neck of the vascular defect, sometimes referred to as a defect spanning portion, need be permeable and/or conducive to thrombus formation in a patient's bloodstream. For such embodiments, that portion of the device that does not span an opening or neck of the vascular defect may be substantially non-permeable or completely permeable with a pore or opening configuration that is too large to effectively promote thrombus formation.

In general, it may be desirable in some cases to use a hollow, thin walled device with a permeable shell of resilient material that may be constrained to a low profile for delivery within a patient. Such a device may also be configured to expand radially outward upon removal of the constraint such that the shell of the device assumes a larger volume and fills or otherwise occludes a vascular defect within which it is deployed. The outward radial expansion of the shell may serve to engage some or all of an inner surface of the vascular defect whereby mechanical friction between an outer surface of the permeable shell of the device and the inside surface of the vascular defect effectively anchors the device within the vascular defect. Some embodiments of such a device may also be partially or wholly mechanically captured within a cavity of a vascular defect, particularly where the defect has a narrow neck portion with a larger interior volume. In order to achieve a low profile and volume for delivery and be capable of a high ratio of expansion by volume, some device embodiments include a matrix of woven or braided filaments that are coupled together by the interwoven structure so as to form a self-expanding permeable shell having a pore or opening pattern between couplings or intersections of the filaments that is substantially regularly spaced and stable, while still allowing for conformity and volumetric constraint.

As used herein, the terms woven and braided are used interchangeably to mean any form of interlacing of filaments to form a mesh structure. In the textile and other industries, these terms may have different or more specific meanings depending on the product or application such as whether an article is made in a sheet or cylindrical form. For purposes of the present disclosure, these terms are used interchangeably.

For some embodiments, three factors may be critical for a woven or braided wire occlusion device for treatment of a patient's vasculature that can achieve a desired clinical outcome in the endovascular treatment of cerebral aneurysms. We have found that for effective use in some applications, it may be desirable for the implant device to have sufficient radial stiffness for stability, limited pore size for near-complete acute (intra-procedural) occlusion and a collapsed profile which is small enough to allow insertion through an inner lumen of a microcatheter. A device with a radial stiffness below a certain threshold may be unstable and may be at higher risk of embolization in some cases. Larger pores between filament intersections in a braided or woven structure may not generate thrombus and occlude a vascular defect in an acute setting and thus may not give a treating physician or health professional such clinical feedback that the flow disruption will lead to a complete and lasting occlusion of the vascular defect being treated. Delivery of a device for treatment of a patient's vasculature through a standard microcatheter may be highly desirable to allow access through the tortuous cerebral vasculature in the manner that a treating physician is accustomed. A detailed discussion of radial stiffness, pore size, and the necessary collapsed profile can be found in US 2017/0095254, which was previously expressly incorporated by reference in its entirety.

As has been discussed, some embodiments of devices for treatment of a patient's vasculature call for sizing the device which approximates (or with some over-sizing) the vascular site dimensions to fill the vascular site. One might assume that scaling of a device to larger dimensions and using larger filaments would suffice for such larger embodiments of a device. However, for the treatment of brain aneurysms, the diameter or profile of the radially collapsed device is limited by the catheter sizes that can be effectively navigated within the small, tortuous vessels of the brain. Further, as a device is made larger with a given or fixed number of resilient filaments having a given size or thickness, the pores or openings between junctions of the filaments are correspondingly larger. In addition, for a given filament size the flexural modulus or stiffness of the filaments and thus the structure decrease with increasing device dimension. Flexural modulus may be defined as the ratio of stress to strain. Thus, a device may be considered to have a high flexural modulus or be stiff if the strain (deflection) is low under a given force. A stiff device may also be said to have low compliance.

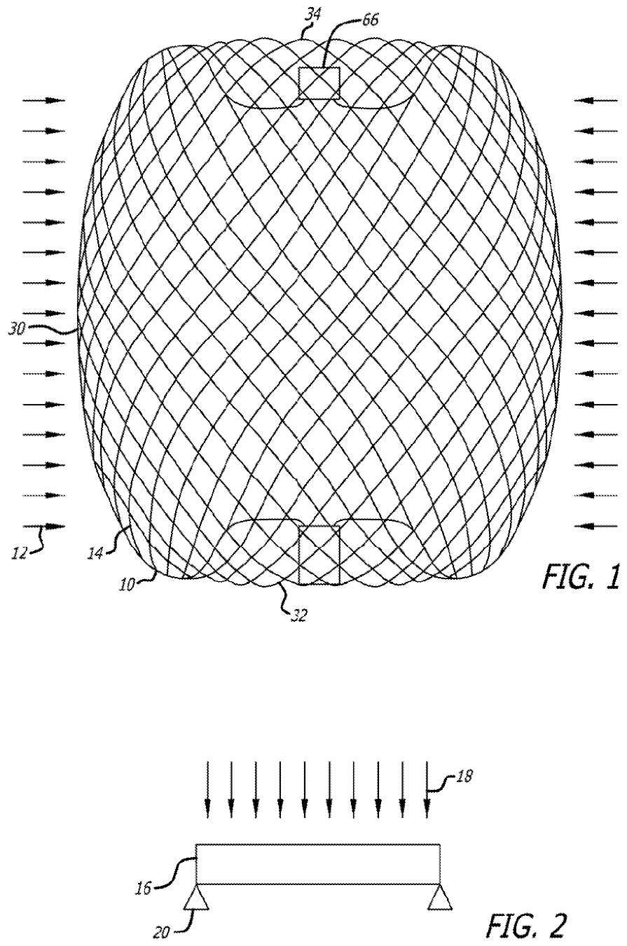

To properly configure larger size devices for treatment of a patient's vasculature, it may be useful to model the force on a device when the device is deployed into a vascular site or defect, such as a blood vessel or aneurysm, that has a diameter or transverse dimension that is smaller than a nominal diameter or transverse dimension of the device in a relaxed unconstrained state. As discussed, it may be advisable to “over-size” the device in some cases so that there is a residual force between an outside surface of the device and an inside surface of the vascular wall. The inward radial force on a device 10 that results from over-sizing is illustrated schematically in FIG. 1 with the arrows 12 in the figure representing the inward radial force. As shown in FIG. 2, these compressive forces on the filaments 14 of the device in FIG. 1 can be modeled as a simply supported beam 16 with a distributed load or force as show by the arrows 18 in the figure. It can be seen from the equation below for the deflection of a beam with two simple supports 20 and a distributed load that the deflection is a function of the length, L to the 4^{th} power:

Deflection of Beam=5FL⁴/384El
where F=force,
L=length of beam,
E=Young's Modulus, and
l=moment of inertia.

Thus, as the size of the device increases and L increases, the compliance increases substantially. Accordingly, an outward radial force exerted by an outside surface of the filaments 14 of the device 10 against a constraining force when inserted into a vascular site such as blood vessel or aneurysm is lower for a given amount of device compression or over-sizing. This force may be important in some applications to assure device stability and to reduce the risk of migration of the device and potential distal embolization.

In some embodiments, a combination of small and large filament sizes may be utilized to make a device with a desired radial compliance and yet have a collapsed profile which is configured to fit through an inner lumen of commonly used microcatheters. A device fabricated with even a small number of relatively large filaments 14 can provide reduced radial compliance (or increased stiffness) compared to a device made with all small filaments. Even a relatively small number of larger filaments may provide a substantial increase in bending stiffness due to change in the moment of Inertia that results from an increase in diameter without increasing the total cross sectional area of the filaments. The moment of inertia (I) of a round wire or filament may be defined by the equation:

I=πd⁴/64
where d is the diameter of the wire or filament.

Since the moment of inertia is a function of filament diameter to the fourth power, a small change in the diameter greatly increases the moment of inertia. Thus, small changes in filament size can have substantial impact on the deflection at a given load and thus the compliance of the device.

Thus, the stiffness can be increased by a significant amount without a large increase in the cross sectional area of a collapsed profile of the device 10. This may be particularly important as device embodiments are made larger to treat large aneurysms. While large cerebral aneurysms may be relatively rare, they present an important therapeutic challenge as some embolic devices currently available to physicians have relatively poor results compared to smaller aneurysms.

As such, some embodiments of devices for treatment of a patient's vasculature may be formed using a combination of filaments 14 with a number of different diameters such as 2, 3, 4, 5 or more different diameters or transverse dimensions. In device embodiments where filaments with two different diameters are used, some larger filament embodiments may have a transverse dimension of about 0.001 inches to about 0.004 inches and some small filament embodiments may have a transverse dimension or diameter of about 0.0004 inches and about 0.0015 inches, more specifically, about 0.0004 inches to about 0.001 inches. The ratio of the number of large filaments to the number of small filaments may be between about 2 and 12 and may also be between about 4 and 8. In some embodiments, the difference in diameter or transverse dimension between the larger and smaller filaments may be less than about 0.004 inches, more specifically, less than about 0.0035 inches, and even more specifically, less than about 0.002 inches.

As discussed above, device embodiments 10 for treatment of a patient's vasculature may include a plurality of wires, fibers, threads, tubes or other filamentary elements that form a structure that serves as a permeable shell. For some embodiments, a globular shape may be formed from such filaments by connecting or securing the ends of a tubular braided structure. For such embodiments, the density of a braided or woven structure may inherently increase at or near the ends where the wires or filaments 14 are brought together and decrease at or near a middle portion 30 disposed between a proximal end 32 and distal end 34 of the permeable shell 40. For some embodiments, an end or any other suitable portion of a permeable shell 40 may be positioned in an opening or neck of a vascular defect such as an aneurysm for treatment. As such, a braided or woven filamentary device with a permeable shell may not require the addition of a separate defect spanning structure having properties different from that of a nominal portion of the permeable shell to achieve hemostasis and occlusion of the vascular defect. Such a filamentary device may be fabricated by braiding, weaving or other suitable filament fabrication techniques. Such device embodiments may be shape set into a variety of three-dimensional shapes such as discussed herein.

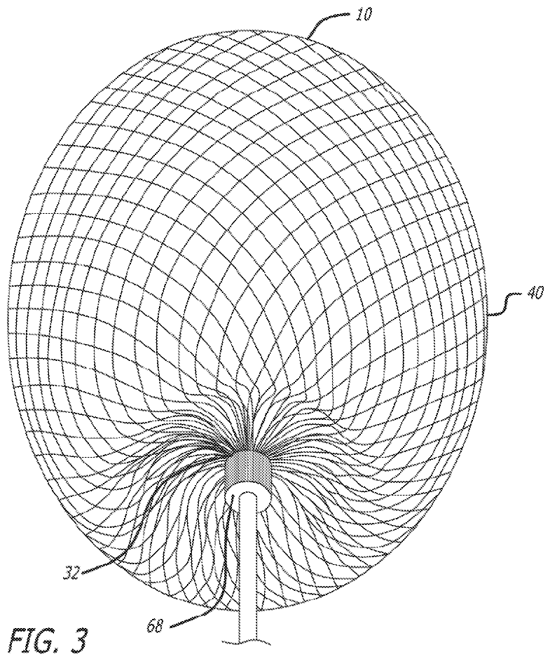

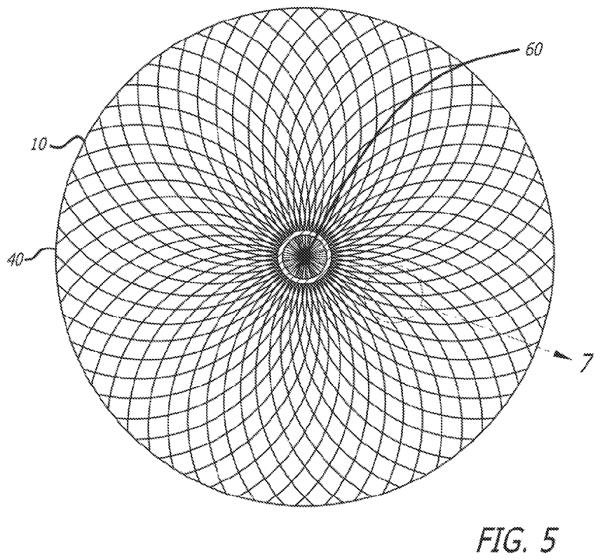

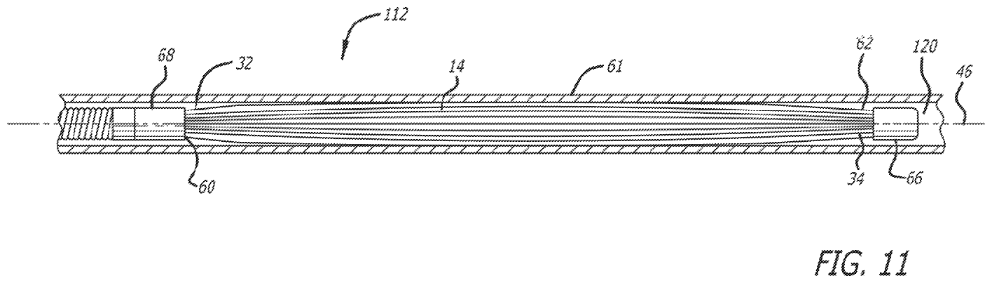

Referring to FIGS. 3-10, an embodiment of a device for treatment of a patient's vasculature 10 is shown. The device 10 includes a self-expanding resilient permeable shell 40 having a proximal end 32, a distal end 34, a longitudinal axis 46 and further comprising a plurality of elongate resilient filaments 14 including large filaments 48 and small filaments 50 of at least two different transverse dimensions as shown in more detail in FIGS. 5, 7, and 18. The filaments 14 have a woven structure and are secured relative to each other at proximal ends 60 and distal ends 62 thereof. The permeable shell 40 of the device has a radially constrained elongated state configured for delivery within a microcatheter 61, as shown in FIG. 11, with the thin woven filaments 14 extending longitudinally from the proximal end 42 to the distal end 44 radially adjacent each other along a length of the filaments.

As shown in FIGS. 3-6, the permeable shell 40 also has an expanded relaxed state with a globular and longitudinally shortened configuration relative to the radially constrained state. In the expanded state, the woven filaments 14 form the self-expanding resilient permeable shell 40 in a smooth path radially expanded from a longitudinal axis 46 of the device between the proximal end 32 and distal end 34. The woven structure of the filaments 14 includes a plurality of openings 64 in the permeable shell 40 formed between the woven filaments. For some embodiments, the largest of said openings 64 may be configured to allow blood flow through the openings only at a velocity below a thrombotic threshold velocity. Thrombotic threshold velocity has been defined, at least by some, as the time-average velocity at which more than 50% of a vascular graft surface is covered by thrombus when deployed within a patient's vasculature. In the context of aneurysm occlusion, a slightly different threshold may be appropriate. Accordingly, the thrombotic threshold velocity as used herein shall include the velocity at which clotting occurs within or on a device, such as device 10, deployed within a patient's vasculature such that blood flow into a vascular defect treated by the device is substantially blocked in less than about 1 hour or otherwise during the treatment procedure. The blockage of blood flow into the vascular defect may be indicated in some cases by minimal contrast agent entering the vascular defect after a sufficient amount of contrast agent has been injected into the patient's vasculature upstream of the implant site and visualized as it dissipates from that site. Such sustained blockage of flow within less than about 1 hour or during the duration of the implantation procedure may also be referred to as acute occlusion of the vascular defect.

As such, once the device 10 is deployed, any blood flowing through the permeable shell may be slowed to a velocity below the thrombotic threshold velocity and thrombus will begin to form on and around the openings in the permeable shell 40. Ultimately, this process may be configured to produce acute occlusion of the vascular defect within which the device 10 is deployed. For some embodiments, at least the distal end of the permeable shell 40 may have a reverse bend in an everted configuration such that the secured distal ends 62 of the filaments 14 are withdrawn axially within the nominal permeable shell structure or contour in the expanded state. For some embodiments, the proximal end of the permeable shell further includes a reverse bend in an everted configuration such that the secured proximal ends 60 of the filaments 14 are withdrawn axially within the nominal permeable shell structure 40 in the expanded state. As used herein, the term everted may include a structure that is everted, partially everted and/or recessed with a reverse bend as shown in the device embodiment of FIGS. 3-6. For such embodiments, the ends 60 and 62 of the filaments 14 of the permeable shell or hub structure disposed around the ends may be withdrawn within or below the globular shaped periphery of the permeable shell of the device.

The elongate resilient filaments 14 of the permeable shell 40 may be secured relative to each other at proximal ends 60 and distal ends 62 thereof by one or more methods including welding, soldering, adhesive bonding, epoxy bonding or the like. In addition to the ends of the filaments being secured together, a distal hub 66 may also be secured to the distal ends 62 of the thin filaments 14 of the permeable shell 40 and a proximal hub 68 secured to the proximal ends 60 of the thin filaments 14 of the permeable shell 40. The proximal hub 68 may include a cylindrical member that extends proximally beyond the proximal ends 60 of the thin filaments so as to form a cavity 70 within a proximal portion of the proximal hub 68. The proximal cavity 70 may be used for holding adhesives such as epoxy, solder or any other suitable bonding agent for securing an elongate detachment tether 72 that may in turn be detachably secured to a delivery apparatus such as is shown in FIG. 11.

For some embodiments, the elongate resilient filaments 14 of the permeable shell 40 may have a transverse cross section that is substantially round in shape and be made from a superelastic material that may also be a shape memory metal. The shape memory metal of the filaments of the permeable shell 40 may be heat set in the globular configuration of the relaxed expanded state as shown in FIGS. 3-6. Suitable superelastic shape memory metals may include alloys such as NiTi alloy and the like. The superelastic properties of such alloys may be useful in providing the resilient properties to the elongate filaments 14 so that they can be heat set in the globular form shown, fully constrained for delivery within an inner lumen of a microcatheter and then released to self expand back to substantially the original heat set shape of the globular configuration upon deployment within a patient's body.

The device 10 may have an everted filamentary structure with a permeable shell 40 having a proximal end 32 and a distal end 34 in an expanded relaxed state. The permeable shell 40 has a substantially enclosed configuration for the embodiments shown. Some or all of the permeable shell 40 of the device 10 may be configured to substantially block or impede fluid flow or pressure into a vascular defect or otherwise isolate the vascular defect over some period of time after the device is deployed in an expanded state. The permeable shell 40 and device 10 generally also has a low profile, radially constrained state, as shown in FIG. 11, with an elongated tubular or cylindrical configuration that includes the proximal end 32, the distal end 34 and a longitudinal axis 46. While in the radially constrained state, the elongate flexible filaments 14 of the permeable shell 40 may be disposed substantially parallel and in close lateral proximity to each other between the proximal end and distal end forming a substantially tubular or compressed cylindrical configuration.

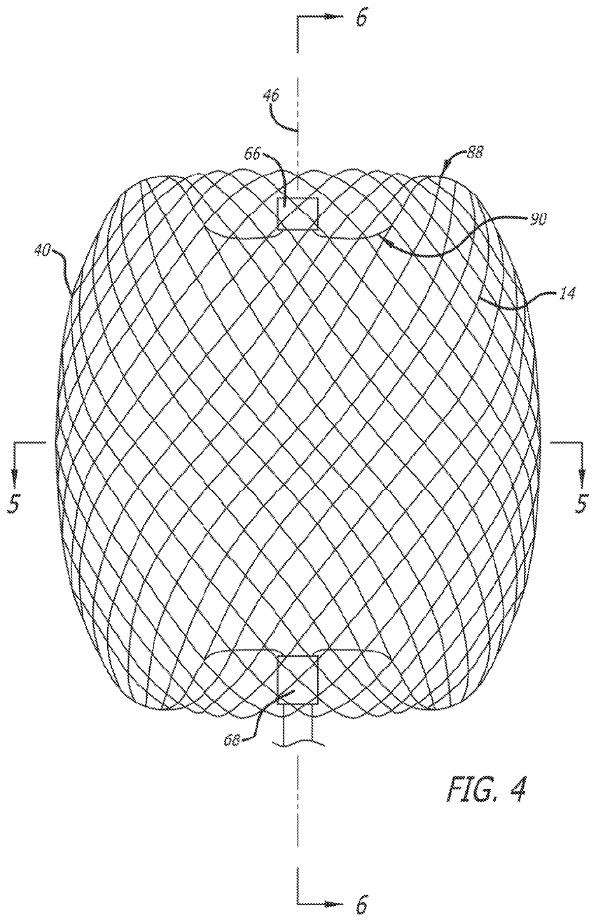

Proximal ends 60 of at least some of the filaments 14 of the permeable shell 40 may be secured to the proximal hub 68 and distal ends 62 of at least some of the filaments 14 of the permeable shell 40 are secured to the distal hub 66, with the proximal hub 68 and distal hub 66 being disposed substantially concentric to the longitudinal axis 46 as shown in FIG. 4. The ends of the filaments 14 may be secured to the respective hubs 66 and 68 by any of the methods discussed above with respect to securement of the filament ends to each other, including the use of adhesives, solder, welding and the like. A middle portion 30 of the permeable shell 40 may have a first transverse dimension with a low profile suitable for delivery from a microcatheter as shown in FIG. 11. Radial constraint on the device 10 may be applied by an inside surface of the inner lumen of a microcatheter, such as the distal end portion of the microcatheter 61 shown, or it may be applied by any other suitable mechanism that may be released in a controllable manner upon ejection of the device 10 from the distal end of the catheter. In FIG. 11 a proximal end or hub 68 of the device 10 is secured to a distal end of an elongate delivery apparatus 111 of a delivery system 112 disposed at the proximal hub 68 of the device 10. Additional details of delivery devices can be found in, e.g., US 2016/0367260, which was previously incorporated by reference in its entirety.

Some device embodiments 10 having a braided or woven filamentary structure may be formed using about 10 filaments to about 300 filaments 14, more specifically, about 10 filaments to about 100 filaments 14, and even more specifically, about 60 filaments to about 80 filaments 14. Some embodiments of a permeable shell 40 may include about 70 filaments to about 300 filaments extending from the proximal end 32 to the distal end 34, more specifically, about 100 filaments to about 200 filaments extending from the proximal end 32 to the distal end 34. For some embodiments, the filaments 14 may have a transverse dimension or diameter of about 0.0008 inches to about 0.004 inches. The elongate resilient filaments 14 in some cases may have an outer transverse dimension or diameter of about 0.0005 inch to about 0.005 inch, more specifically, about 0.001 inch to about 0.003 inch, and in some cases about 0.0004 inches to about 0.002 inches. For some device embodiments 10 that include filaments 14 of different sizes, the large filaments 48 of the permeable shell 40 may have a transverse dimension or diameter that is about 0.001 inches to about 0.004 inches and the small filaments 50 may have a transverse dimension or diameter of about 0.0004 inches to about 0.0015 inches, more specifically, about 0.0004 inches to about 0.001 inches. In addition, a difference in transverse dimension or diameter between the small filaments 50 and the large filaments 48 may be less than about 0.004 inches, more specifically, less than about 0.0035 inches, and even more specifically, less than about 0.002 inches. For embodiments of permeable shells 40 that include filaments 14 of different sizes, the number of small filaments 50 of the permeable shell 40 relative to the number of large filaments 48 of the permeable shell 40 may be about 2 to 1 to about 15 to 1, more specifically, about 2 to 1 to about 12 to 1, and even more specifically, about 4 to 1 to about 8 to 1.

The expanded relaxed state of the permeable shell 40, as shown in FIG. 4, has an axially shortened configuration relative to the constrained state such that the proximal hub 68 is disposed closer to the distal hub 66 than in the constrained state. Both hubs 66 and 68 are disposed substantially concentric to the longitudinal axis 46 of the device and each filamentary element 14 forms a smooth arc between the proximal and distal hubs 66 and 68 with a reverse bend at each end. A longitudinal spacing between the proximal and distal hubs 66 and 68 of the permeable shell 40 in a deployed relaxed state may be about 25 percent to about 75 percent of the longitudinal spacing between the proximal and distal hubs 66 and 68 in the constrained cylindrical state, for some embodiments. The arc of the filaments 14 between the proximal and distal ends 32 and 34 may be configured such that a middle portion of each filament 14 has a second transverse dimension substantially greater than the first transverse dimension.

For some embodiments, the permeable shell 40 may have a first transverse dimension in a collapsed radially constrained state of about 0.2 mm to about 2 mm and a second transverse dimension in a relaxed expanded state of about 4 mm to about 30 mm. For some embodiments, the second transverse dimension of the permeable shell 40 in an expanded state may be about 2 times to about 150 times the first transverse dimension, more specifically, about 10 times to about 25 times the first or constrained transverse dimension. A longitudinal spacing between the proximal end 32 and distal end 34 of the permeable shell 40 in the relaxed expanded state may be about 25% percent to about 75% percent of the spacing between the proximal end 32 and distal end 34 in the constrained cylindrical state. For some embodiments, a major transverse dimension of the permeable shell 40 in a relaxed expanded state may be about 4 mm to about 30 mm, more specifically, about 9 mm to about 15 mm, and even more specifically, about 4 mm to about 8 mm.

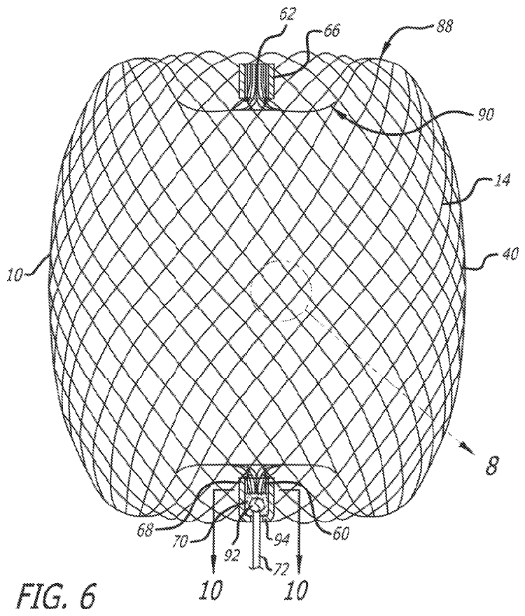

An arced portion of the filaments 14 of the permeable shell 40 may have a sinusoidal-like shape with a first or outer radius 88 and a second or inner radius 90 near the ends of the permeable shell 40 as shown in FIG. 6. This sinusoid-like or multiple curve shape may provide a concavity in the proximal end 32 that may reduce an obstruction of flow in a parent vessel adjacent a vascular defect. For some embodiments, the first radius 88 and second radius 90 of the permeable shell 40 may be between about 0.12 mm to about 3 mm. For some embodiments, the distance between the proximal end 32 and distal end 34 may be less than about 60% of the overall length of the permeable shell 40 for some embodiments. Such a configuration may allow for the distal end 34 to flex downward toward the proximal end 32 when the device 10 meets resistance at the distal end 34 and thus may provide longitudinal conformance. The filaments 14 may be shaped in some embodiments such that there are no portions that are without curvature over a distance of more than about 2 mm. Thus, for some embodiments, each filament 14 may have a substantially continuous curvature. This substantially continuous curvature may provide smooth deployment and may reduce the risk of vessel perforation. For some embodiments, one of the ends 32 or 34 may be retracted or everted to a greater extent than the other so as to be more longitudinally or axially conformal than the other end.

The first radius 88 and second radius 90 of the permeable shell 40 may be between about 0.12 mm to about 3 mm for some embodiments. For some embodiments, the distance between the proximal end 32 and distal end 34 may be more than about 60% of the overall length of the expanded permeable shell 40. Thus, the largest longitudinal distance between the inner surfaces may be about 60% to about 90% of the longitudinal length of the outer surfaces or the overall length of device 10. A gap between the hubs 66 and 68 at the proximal end 32 and distal end 34 may allow for the distal hub 66 to flex downward toward the proximal hub 68 when the device 10 meets resistance at the distal end and thus provides longitudinal conformance. The filaments 14 may be shaped such that there are no portions that are without curvature over a distance of more than about 2 mm. Thus, for some embodiments, each filament 14 may have a substantially continuous curvature. This substantially continuous curvature may provide smooth deployment and may reduce the risk of vessel perforation. The distal end 34 may be retracted or everted to a greater extent than the proximal end 32 such that the distal end portion of the permeable shell 40 may be more radially conformal than the proximal end portion. Conformability of a distal end portion may provide better device conformance to irregular shaped aneurysms or other vascular defects. A convex surface of the device may flex inward forming a concave surface to conform to curvature of a vascular site.

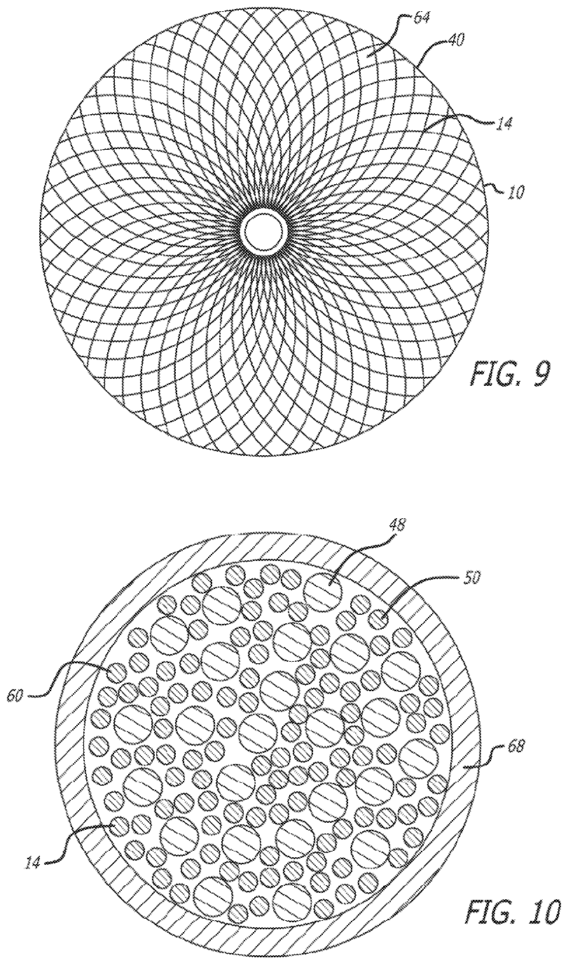

FIG. 10 shows an enlarged view of the filaments 14 disposed within a proximal hub 68 of the device 10 with the filaments 14 of two different sizes constrained and tightly packed by an outer ring of the proximal hub 68. The tether member 72 may optionally be disposed within a middle portion of the filaments 14 or within the cavity 70 of the proximal hub 68 proximal of the proximal ends 60 of the filaments 14 as shown in FIG. 6. The distal end of the tether 72 may be secured with a knot 92 formed in the distal end thereof which is mechanically captured in the cavity 70 of the proximal hub 68 formed by a proximal shoulder portion 94 of the proximal hub 68. The knotted distal end 92 of the tether 72 may also be secured by bonding or potting of the distal end of the tether 72 within the cavity 70 and optionally amongst the proximal ends 60 of the filaments 14 with mechanical compression, adhesive bonding, welding, soldering, brazing or the like. The tether embodiment 72 shown in FIG. 6 has a knotted distal end 92 potted in the cavity of the proximal hub 68 with an adhesive. Such a tether 72 may be a dissolvable, severable or releasable tether that may be part of a delivery apparatus 111 used to deploy the device 10 as shown in FIG. 11 and FIGS. 23-26. FIG. 10 also shows the large filaments 48 and small filaments 50 disposed within and constrained by the proximal hub 68 which may be configured to secure the large and small filaments 48 and 50 in place relative to each other within the outer ring of the proximal hub 68.

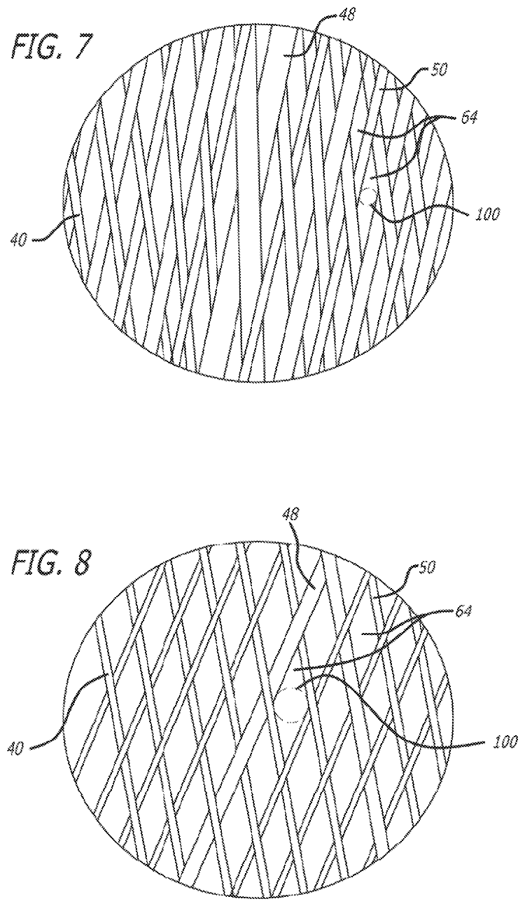

FIGS. 7 and 8 illustrate some configuration embodiments of braided filaments 14 of a permeable shell 40 of the device 10 for treatment of a patient's vasculature. The braid structure in each embodiment is shown with a circular shape 100 disposed within a pore 64 of a woven or braided structure with the circular shape 100 making contact with each adjacent filament segment. The pore opening size may be determined at least in part by the size of the filament elements 14 of the braid, the angle overlapping filaments make relative to each other and the picks per inch of the braid structure. For some embodiments, the cells or openings 64 may have an elongated substantially diamond shape as shown in FIG. 7, and the pores or openings 64 of the permeable shell 40 may have a substantially more square shape toward a middle portion 30 of the device 10, as shown in FIG. 8. The diamond shaped pores or openings 64 may have a length substantially greater than the width particularly near the hubs 66 and 68. In some embodiments, the ratio of diamond shaped pore or opening length to width may exceed a ratio of 3 to 1 for some cells. The diamond-shaped openings 64 may have lengths greater than the width thus having an aspect ratio, defined as Length/Width of greater than 1. The openings 64 near the hubs 66 and 68 may have substantially larger aspect ratios than those farther from the hubs as shown in FIG. 7. The aspect ratio of openings 64 adjacent the hubs may be greater than about 4 to 1. The aspect ratio of openings 64 near the largest diameter may be between about 0.75 to 1 and about 2 to 1 for some embodiments. For some embodiments, the aspect ratio of the openings 64 in the permeable shell 40 may be about 0.5 to 1 to about 2 to 1.

The pore size defined by the largest circular shapes 100 that may be disposed within openings 64 of the braided structure of the permeable shell 40 without displacing or distorting the filaments 14 surrounding the opening 64 may range in size from about 0.005 inches to about 0.01 inches, more specifically, about 0.006 inches to about 0.009 inches, even more specifically, about 0.007 inches to about 0.008 inches for some embodiments. In addition, at least some of the openings 64 formed between adjacent filaments 14 of the permeable shell 40 of the device 10 may be configured to allow blood flow through the openings 64 only at a velocity below a thrombotic threshold velocity. For some embodiments, the largest openings 64 in the permeable shell structure 40 may be configured to allow blood flow through the openings 64 only at a velocity below a thrombotic threshold velocity. As discussed above, the pore size may be less than about 0.016 inches, more specifically, less than about 0.012 inches for some embodiments. For some embodiments, the openings 64 formed between adjacent filaments 14 may be about 0.005 inches to about 0.04 inches.

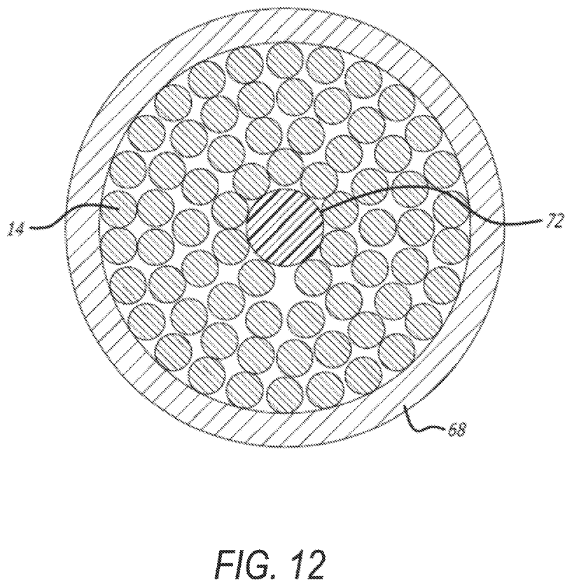

FIG. 12 illustrates in transverse cross section an embodiment of a proximal hub 68 showing the configuration of filaments which may be tightly packed and radially constrained by an inside surface of the proximal hub 68. In some embodiments, the braided or woven structure of the permeable shell 40 formed from such filaments 14 may be constructed using a large number of small filaments. The number of filaments 14 may be greater than 125 and may also be between about 80 filaments and about 180 filaments. As discussed above, the total number of filaments 14 for some embodiments may be about 70 filaments to about 300 filaments, more specifically, about 100 filaments to about 200 filaments. In some embodiments, the braided structure of the permeable shell 40 may be constructed with two or more sizes of filaments 14. For example, the structure may have several larger filaments that provide structural support and several smaller filaments that provide the desired pore size and density and thus flow resistance to achieve a thrombotic threshold velocity in some cases. For some embodiments, small filaments 50 of the permeable shell 40 may have a transverse dimension or diameter of about 0.0006 inches to about 0.002 inches for some embodiments and about 0.0004 inches to about 0.001 inches in other embodiments. The large filaments 48 may have a transverse dimension or diameter of about 0.0015 inches to about 0.004 inches in some embodiments and about 0.001 inches to about 0.004 inches in other embodiments. The filaments 14 may be braided in a plain weave that is one under, one over structure (shown in FIGS. 7 and 8) or a supplementary weave; more than one warp interlace with one or more than one weft. The pick count may be varied between about 25 and 200 picks per inch (PPI).

### Multiple Layer Devices

The best balance of deliverability, sufficient radial force, and high metal surface area is often achieved with a braid comprising a few relatively large wires for structural support combined with many small wires for stasis. This is because implant stiffness is proportional to d⁴ (where d=wire diameter), whereas the collapsed diameter (and therefore, collapsed area and crossing profile) is proportional to d². This is demonstrated more clearly in Table 1 below, which compares various braids: a 100-wire braid where all wires have (a relative) diameter of 1 (arbitrary unit), another braid with 6 “structural” wires of diameter 2 combined with 300 small wires with diameter equal to ½, and a 106-wire braid incorporating 6 “structural” wires of diameter 2 and 100 small wires of diameter ½ (units in this case are relative dimensions). Relative stiffness values are comparable for all three of these braids so deployment forces and in situ stability should be very similar. Braid 2, however, has many more wires, e.g., compared to Braid 1, and can provide much better stasis. Braid 3 has approximately the same number of total wires as Braid 1 but has a much smaller collapsed area and should therefore fit through a smaller catheter. Thus, Braids 2 and 3 offer significant advantages relative to the “baseline” Braid 1, where all wires are equal in size.

TABLE 1
Braid Configuration

| | | Relative |
|---|---|---|
| | Relative | Collapsed |
| | Stiffness | Area |
| Braid 1: 100 wires of diameter 1 | 100 | 100 |
| Braid 2: 6 wires of diameter 2 + 300 wires of | 115 | 99 |
| diameter ½ | | |
| Braid 3: 6 wires of diameter 2 + 100 wires of | 102 | 49 |
| diameter½ | | |

Occlusion implants made from single layer braids that incorporate a mixture of filament sizes, e.g., a few large and numerous small filaments, may have limitations. In some instances, the large wires may distort the braid structure and adversely impact the ability of the braid to recapture symmetrically within the catheter (leading to high recapture forces). This results from local “warping” of the cells due to the relatively stiffer large wires and from uneven wire bunching during collapse. These issues may occur where the “wire size disparity,” or ratio of large wire diameter to small wire diameter, is greater than about 1.5.

Occlusion implants with multiple layers can solve issues caused by wire size disparity by having a “structural layer” with fewer large wires, which provide structural support for the implants, and a “stasis layer” made from a plurality of small wires, which interfere with blood flow to help promote stagnation or clotting over time. Dual layer devices, however, can have certain disadvantages depending on whether the structural layer is the inside or outside layer. Some of the disadvantages to dual layer implants and flow diverters are listed in Table 2 below.

TABLE 2
Potential Disadvantages of Two Layer Implants

| | Occlusion Implant for | |
|---|---|---|
| Configuration | Aneurysms | Flow Diverters |
| Structural | Requires 3 marker bands/hubs | Additional manufacturing steps required |
| layer inside | Higher delivery friction | to attach layers |
| | | Higher delivery friction |
| | | Requires precise matching of braid |
| | | angles |
| Structural | Requires 3 marker bands/hubs | Additional manufacturing steps required |
| Layer outside | Soft inner layer may not fully | to attach layers |
| | open | Possibility of inner layer separation |
| | Stiffer distal tip | Requires precise matching of braid |
| | | angles |

The use of three marker bands in occlusion implants (e.g., one proximal marker band and two distal marker bands (one for each layer)) can be problematic because the middle marker band may not be perfectly aligned with the distal marker band, resulting in asymmetric collapse (“cobra-heading”) and/or protrusion of the middle marker band through the outer layer. Incorporating three marker bands is also more difficult during manufacturing because it requires more assembly steps.

Note though the term “marker bands” are used herein to discuss various embodiments, these elements are configured as hubs which are generally a junction where the wires forming a device, device section, or device layer are attached together along the hub (e.g., cylindrical structure). Often it is beneficial that this hub is radiopaque to aid in visualization, and as such the hub often takes on the form of a marker band. In this way, the marker band is inclusive of a hub concept.

Moreover, the ordering of the layers may present issues. In an occlusion implant, if the structural layer is inside of the stasis layer, it can facilitate expansion of (e.g., “push open”) the relatively soft stasis layer during deployment. This configuration, however, may have higher delivery forces during advancement through the microcatheter because the soft outer layer may be more likely to deform or fold on itself, and the high wire count may lead to many more points of contact with the catheter inner surface causing higher friction.

Additionally, the extremely small wires of the outer stasis layer may break during delivery as they shear against the catheter inner lumen. Conversely, an implant in which the structural layer is the outer layer circumvents these friction/wire break issues, but may result in a stiffer distal tip. Furthermore, the relatively soft inner layer might not fully open during deployment.

Dual layer intralumenal flow diverter implants may have similar issues. For example, the structural and stasis layers may need to be secured together after braiding and heat setting (with a “woven” filament, for example), which is more difficult during manufacturing. Furthermore, braid angles for the inner and outer layers may need to be precisely matched or the implant may not collapse evenly. As with the occlusion implants, an inner structural layer may facilitate expansion of the soft outer stasis layer during deployment but may result in higher friction due to the softness and higher surface area of the stasis layer. Furthermore, separation of the inner and outer layers, especially near the distal and proximal ends of the implant, may be a concern.

### Three Layer Implants

Occlusion Implants

Implants with three or more layers can combine the advantages obtained with separate structural and stasis layers while circumventing many of the problems identified above. An occlusion implant with an outer “fold-over” (dual layer) braid for, e.g., stasis, combined with a single inner layer, e.g., structural layer, may include only two marker bands. Moreover, the number of wires in contact with the catheter during delivery may be lower than with the equivalent dual layer design because only the “folded over” layer is in contact with the inner lumen of the catheter during delivery. Furthermore, it may be possible to utilize only a proximal marker band if the distal end of the structural layer is composed of looped wires such that the distal end may have a small opening, but be sufficiently gathered without a marker band. A distal marker band may not be necessary.

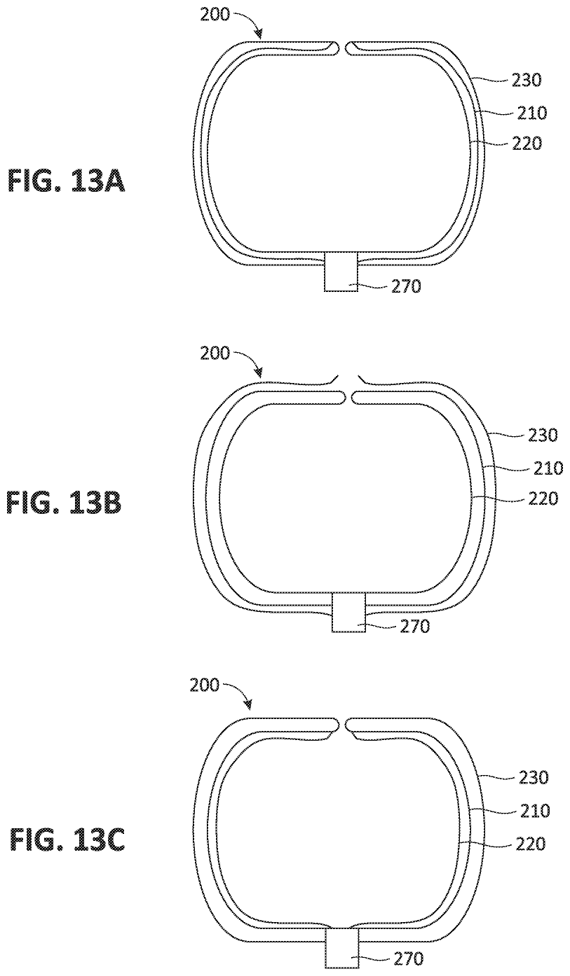

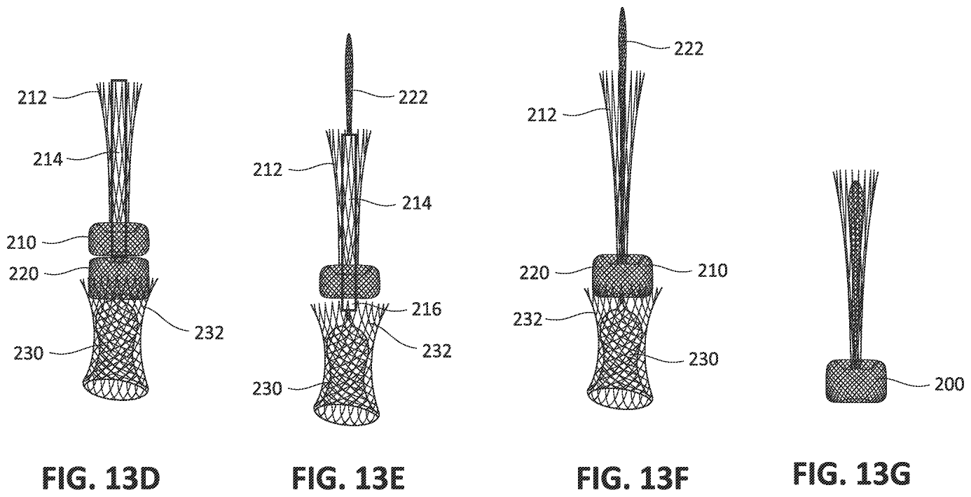

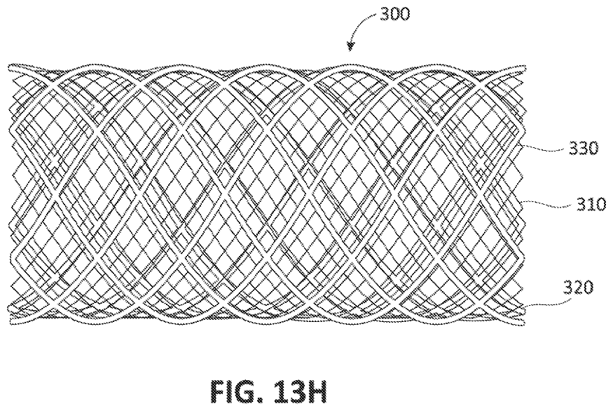

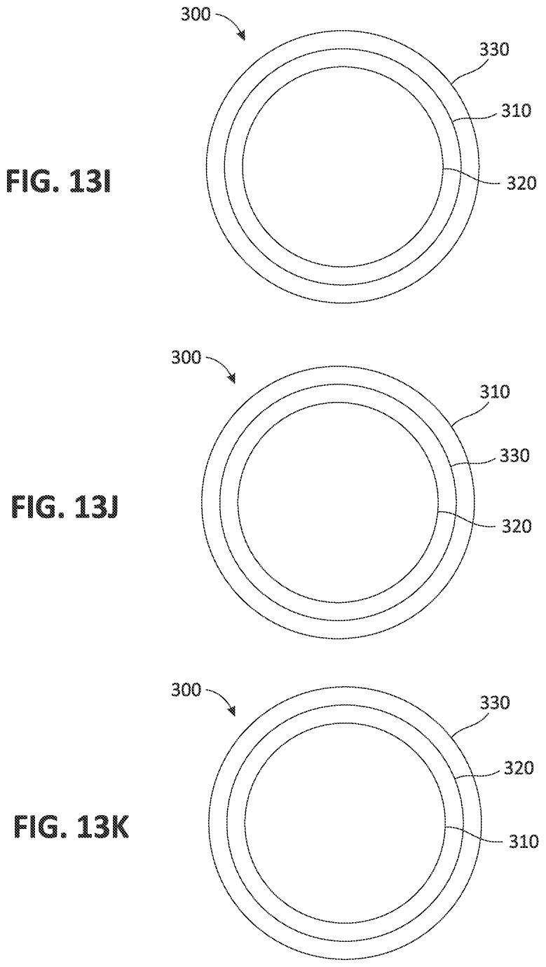

As seen in FIGS. 13A-13C and listed in Table 3 below, three-layer implants 200 that include first and second layers 220, 230 made from a “fold-over” mesh and a single mesh layer 210 can have six possible permutations or arrangements. A structural layer braid with relatively few, large wires may be folded over itself. A high wire count (small wire) stasis layer may be located between the two structural layers (configuration E), as the innermost layer (configuration C), or as the outermost layer (configuration D). Alternatively, a stasis layer with a high wire count (small wire) may be folded over itself. A structural layer braid with relatively few, large wires may be located between the two stasis layers (configuration F), as the innermost layer (configuration A), or as the outermost layer (configuration B).

TABLE 3
Possible Configurations for Three-Layer Implants

| | | Inner | Middle | Outer |
|---|---|---|---|---|
| Configuration | Description | Layer | Layer | Layer |
| A | structural layer completely | structural | stasis | stasis |
| | inside of folded over stasis | | | |
| | layers | | | |
| B | structural layer completely | stasis | stasis | structural |
| | outside folded over stasis layers | | | |
| C | stasis layer completely inside | stasis | structural | structural |
| | folded over structural layers | | | |
| D | stasis layer completely outside | structural | structural | stasis |
| | folded over structural layers | | | |
| E | stasis layer captured within | structural | stasis | structural |
| | folded over structural layers | | | |
| F | structural layer captured within | stasis | structural | stasis |
| | folded over stasis layers | | | |

With respect to the assembly of configuration E, a structural layer braid with relatively few, large wires may be folded over itself, heat set, and then pulled open such that the two layers are in series rather than parallel. A high wire count (small wire) stasis layer can then be advanced between the layers, after which the outermost structural layer can be pulled back again (over the middle stasis and inner structural layer). The resulting (partially assembled) (e.g., FIG. 13D) includes 3 layers—inner and outer structural layers with a middle high-wire count stasis layer. Configuration E may include only a single marker band at a proximal end, and therefore has a softer distal tip (that does not include a marker band). Furthermore, the middle layer may be constructed of extremely small, fragile and soft (low radial force) filaments because they are protected from damage during advancement and retrieval through the delivery catheter by the outer layer. Additionally, any issues with deployment of the softer stasis layer may be eliminated in configuration E because the stasis layer is pushed open during deployment by the inner structural layer. This design may therefore allow for a much higher wire count while maintaining a low profile, appropriate radial strength, and low delivery forces. The inverse 3-layer design is also possible (configuration F)—a structural layer captured within high wire count “folded over” stasis layers.

Generally speaking, in order to prepare a three-layer implant 200, the layers 220, 230 of the permeable shell must first be formed. In order to shape these layers, various molds (e.g., three molds) each having a unique shape corresponding to each separate layer are used. In order to create this tri-layer design, one or more tubular bands are used to create a constriction where the three layers may be transitioned to achieve the tri-layer design. Generally, a tubular mesh is used as a basic implant layer, where the mesh and bands as described are then placed over the various mold shapes to the create different shaped sections. Where the tubular bands as described are used to create transition sections, the sections can then be enveloped backwards to create a tri-layer layer implant.

The single layer permeable shell 210 may be made according to similar steps described above, without inverting the mesh brad tube to make two layers. After the mold has been heat set and removed, the marker band may be removed and/or the distal end of the single layer permeable shell have may be cut, such that the distal end of the single layer permeable shell has an opening. A mesh tail 212 extends from the proximal end of single layer permeable shell 210.

Assembly of the three-layer implant, where the single layer permeable shell 210 is sandwiched between the fold-over layers 220, 230 (see FIG. 13A), is depicted in FIGS. 13D-13G. Each of the single layer permeable shell 210 and fold-over layers has a mesh tail that can be used to assemble the layers together. As seen in FIG. 13D, the single layer permeable shell 210 may be loaded over an introducer sheath 214, such that the introducer sheath extends through the mesh tail 212 and interior cavity of the single layer permeable shell 210 and out of the distal opening. As seen in FIG. 13E, the mesh tail 222 of permeable shell 220 may be inserted into and pulled proximally through the lumen of the introducer sheath 214 such that permeable shell 220 is pulled into the lumen of introducer sheath 214. The distal end 216 of introducer sheath 214 may be moved proximally relative to single layer permeable shell 210 until the distal end 216 is positioned in the inner cavity of single layer permeable shell 210. The introducer sheath 214 may then be withdrawn proximally relative to mesh tail 222, such that fold-over permeable shell 220 deploys within the inner cavity of single layer permeable shell 210, and the introducer sheath 214 may then be removed (see FIG. 13F). The top layer (second portion of fold-over) 230 can then be folded back over (inverted) the single layer permeable shell 210 and the permeable shell 220 (see FIG. 13G) to form implant device 200.

Assembly of the three-layer implant where the single layer permeable shell 210 is the outermost layer (see FIG. 13B) may be accomplished in a similar manner. The fold-over layer permeable shell 230 can be inverted to cover permeable shell 220. Mesh tails 222 and 232 can then be inserted into the lumen of introducer sheath 214 and pulled proximally such that permeable shells 220, 230 are also pulled into the lumen of the introducer sheath 214. The introducer can then be inserted into the mesh tail 212 of single permeable layer 210 and the introducer sheath 214 can be advanced until the distal end 216 is in the inner cavity of single permeable layer 210. Introducer sheath 214 can then be withdrawn proximally relative to mesh tails 222, 232, such that the folded over permeable shells 220, 230 expand to their expanded state in the inner cavity of single permeable layer 210 to form implant device 200.

Assembly of the three-layer implant where the single layer permeable shell 210 is the innermost layer (see FIG. 13C) can be accomplished in a similar manner. The mesh tail 212 of the single layer permeable shell 210 can be pulled proximally through the lumen of introducer sheath 214 such that the single layer permeable shell 210 is also pulled into the lumen of the introducer sheath 214. The introducer sheath 214 can then be inserted into the lumen of the mesh tail 222 of the first portion of the permeable shell 220. Once the distal end 216 of the introducer sheath 214 is in the inner cavity of the fold-over permeable shell 220, the introducer sheath 214 can be withdrawn proximally relative to the mesh tail 212, such that the single layer permeable shell 210 expands to its expanded state in the inner cavity of permeable shell 220. The mesh tail 232 can then be pulled proximally such that the fold-over layer permeable shell 230 inverts and covers permeable shell 220 and single layer permeable shell 210. Alternatively, the fold-over layer permeable shell 230 can be inverted to cover permeable shell 220 before the single layer permeable shell is inserted and deployed within the inner cavity of permeable shell 220.

As discussed previously, any of these layers may be a stasis or structural layer. For occlusion implants, the structural layer may be made from between about 4 and about 48 wires, alternatively between about 4 and about 40 wires, alternatively between about 4 and about 36 wires, alternatively between about 8 and about 40 wires, alternatively between about 4 and about 36 wires, with diameters of about 0.001″—about 0.004″, depending on implant size. The wires of the structural layer may be braided. Generally, lower wire counts may be used with larger wire diameters. The stasis layer may be braided with about 36 to about 216 wires, alternatively about 36 to about 144 wires, with diameters of about 0.00025″—about 0.00075″, depending on implant size. For both structural and stasis layers, the number and size of wires may depend on which layer gets folded over (because folding over will effectively double the number of wires from that braid layer in the final device). Table 4 (below) lists some exemplary wire combinations for the structural and stasis layers for an occlusion device having a 7 mm diameter. The wires or filaments used to make the mesh braids may be made of super-elastic (e.g., nitinol) and/or composite super-elastic/radiopaque (nitinol-platinum “DFT”) material. The plurality of filaments that make up the mesh may be made from nitinol, stainless steel, drawn filled tubing (e.g., platinum or tantalum core with a nitinol jacket), platinum, platinum alloys such as platinum/tungsten, or a mixture thereof. Suitable materials for constructing mesh implants are described in US 2017/0095254, US 2016/0249934, US 2016/0367260, US 2016/0249937, and US 2018/0000489, all of which are hereby expressly incorporated by reference in their entirety for all purposes.

TABLE 4
Possible wire combinations for a 7 mm occlusion implant.

| 7 mm WEB Configuration | Structural Layer | Stasis layer |
|---|---|---|
| Structural layer inside of, | 8 wires × 0.00175″ | 72 wires × 0.00075″ folded |
| outside of, or within folded | | over (144 wires total) |
| over stasis layer | 8 wires × 0.00175″ | 36 wires × 0.00075″ + 36 |
| | | wires × 0.0005″ folded over |
| | | (144 wires total) |
| | 36 wires × 0.00125″ | 54 wires × 0.0005″ folded |
| | | over (108 wires total) |
| Stasis layer inside of, outside | 36 wires × 0.001″ + 18 | 72 × 0.0005″ wires |
| of, or within folded over | wires × 0.00075″ folded | |
| structural layer | over (108 wires total) | |

Flow Diverters

Many of the same concepts described above with respect to occlusion implants may be used with flow diverters. As illustrated below in FIGS. 13H and 13I, a flow diverter 300 may be constructed with a middle stasis layer 310 made up of many small wires captured within “folded over” structural layers 320, 330. This may be advantageous relative to dual layer flow diverters (see Table 2) because such an arrangement maintains the deliverability of a low wire count outer layer while eliminating the concern about layers separating. Additionally, such an implant may be easier to manufacture because the middle layer does not need to be secured to the structural layer because it is captured between the structural layers. The middle layer may also be slightly shorter than the folded over structural layers so that the braid angles do not need to match precisely. As with the occlusion implants, the inverse 3-layer configuration is also possible (structural layer captured within a folded over stasis layer) and any of the other possible permutations (single layer 310 as the innermost (see FIG. 13K) or the single layer 310 as the outermost layer (see FIG. 13J)) may be made.

For flow diverter implants, the structural layer may include between about 4 and about 18 wires. The wires or filaments for the structural layer may have a diameter of about 0.00175″—about 0.0035″. The stasis layer(s) may be made of about 24 to about 144 wires. The wires or filaments for the stasis layer may have a diameter of about 0.00025″—about 0.001″. As with occlusion implants, super-elastic (e.g., nitinol) and/or composite super-elastic/radiopaque (nitinol-platinum “DFT”) material. The plurality of filaments that make up the mesh may be made from nitinol, stainless steel, drawn filled tubing (e.g., platinum or tantalum core with a nitinol jacket), platinum, platinum alloys such as platinum/tungsten, or a mixture thereof. Suitable materials for constructing mesh implants are described in US 2017/0095254, US 2016/0249934, US 2016/0367260, US 2016/0249937, and US 2018/0000489, all of which were previously expressly incorporated by reference in their entirety for all purposes.

For both occlusion and flow diverter implants, flat wire might be used to increase surface area of the stasis layer. Additionally, 4 or more layers could be used if desired.

Reinforced Occlusion Implants with Soft Distal Ends

In some embodiments, occlusion implants with a softer distal end may be made by eliminating the distal maker band, which may cause damage to the aneurysm wall and could potentially lead to rupture of the aneurysm. Although removal of the distal marker band in the fold-over implant designs discussed with respect to other embodiments accomplishes this goal of a softer, atraumatic distal end, the occlusion implants may have less radial stiffness. This decrease in radial stiffness may cause devices to have more difficulty opening or “flowering” as well. In particular, softer implants may twist or fold over themselves and not fully deploy against the wall of the aneurysm or parent artery.

The occlusion implants of the embodiments described in this application may have expanded states that can have a substantially globular, spherical, barrel, or cylindrical shape. Alternatively, the expanded state can have a shape similar to a pumpkin. The proximal and/or distal ends may be recessed or may be substantially flat. The distal and proximal ends of the occlusion implants (e.g., where the wires bend (e.g., about 90°) and transition from the substantially flat distal and proximal ends to the middle region) contribute nearly all of the radial stiffness to the implant, wherein the middle region (forming the substantially vertical or partially curved walls of the expanded state) are almost immeasurably soft. Similarly, with stents and flow diverters, the radial strength is more uniform along the length but devices may have issues fully opening (especially if the stent/flow diverter is relatively long).

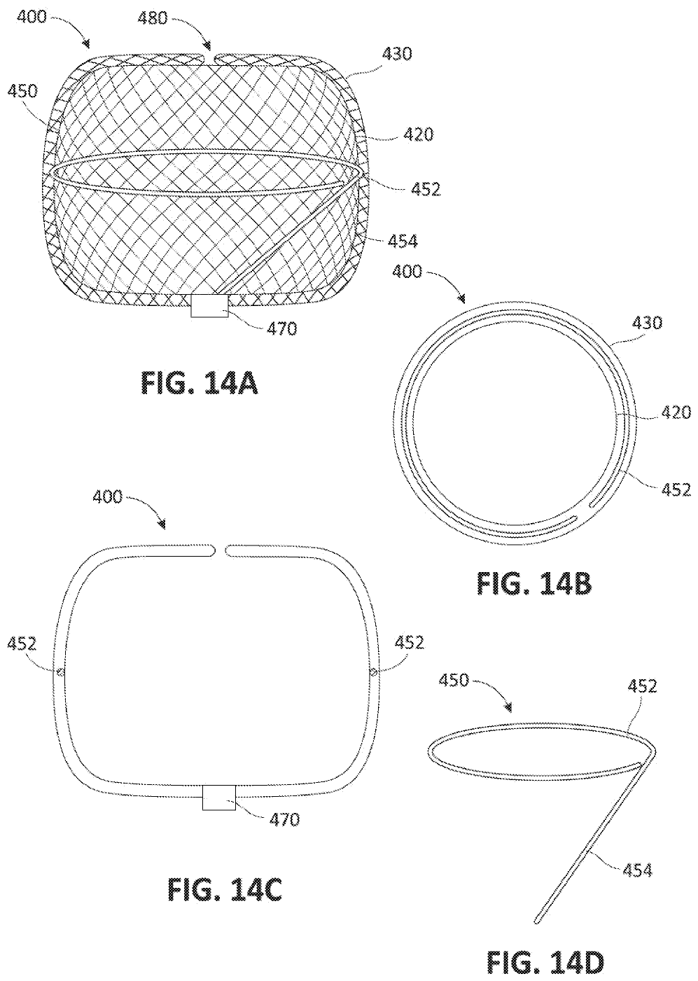

In one embodiment, an implant may include an expanding belt-like structure to assist in the deployment of the implant. As seen in FIGS. 14A-14D, occlusion implant 400 may include first 420 and second 430 mesh layers formed by folding over a mesh tube as described previously. The first and second ends of each of the plurality of filaments that form the first 420 and second 430 mesh layers may be gathered in a proximal hub 470, which may also be a radiopaque marker. The distal end of implant 400 may have an opening 480 that communicates with an inner cavity defined by an inner surface of the first (inner) layer 420, and may not include a distal marker band. The soft, atraumatic distal end may easily conform to the anatomy of the aneurysm in which it is implanted. Additional radial stiffness can be added to implant 400 by including a reinforcing belt 450 that extends around a perimeter of the implant 400 between the first 420 and second 430 mesh layers and can assist in expansion of implant 400.

As seen in FIG. 14D, a reinforcing belt 450 may include a loop portion 452 and a non-loop or substantially straight portion 454. The loop portion 452 may extend around a perimeter of the implant 400 between the first 420 and second 430 mesh layers. The substantially straight portion 454 may extend from the loop portion to the proximal hub 470, where an end of the substantially straight portion may be captured in the proximal hub with the first and second ends of the plurality of filaments that form the first 420 and second 430 mesh layers. The reinforcing belt 450 may be positioned at approximately half the height of the expanded state of the implant 400, e.g., an equatorial belt. Alternatively, the reinforcing belt 450 may be positioned at a location between about 25%-75%, alternatively between about 40%-60% of the total height of expanded state of the implant 400, from the proximal end of the implant.

The reinforcing belt 450 may be made from a super-elastic material, e.g., nitinol wire, which is heat set into a looped configuration. The diameter of the wire itself may be between about 0.002″ to about 0.005″, depending on radial force requirements and collapsed profile space constraints. The reinforcing belt 450 may be heat set such that in its expanded state, a plane defined by the loop 252 is approximately perpendicular to a longitudinal axis of the implant 400 extending from the proximal hub 470 to the distal opening 480. The wire length and heat set diameter may vary based on the size and diameter of the implant. The wire may be heat set into a loop as a single wire. Alternatively, two smaller wires may be heat set as two half loops. Alternatively, various other shapes, e.g., triangular or square could be heat set. Moreover, a plurality of loops, e.g., at least 2, at least 3, at least 4, at least 5 or more hoops, or a spiral or spring-like shape may be used to assist in expansion of the implant.

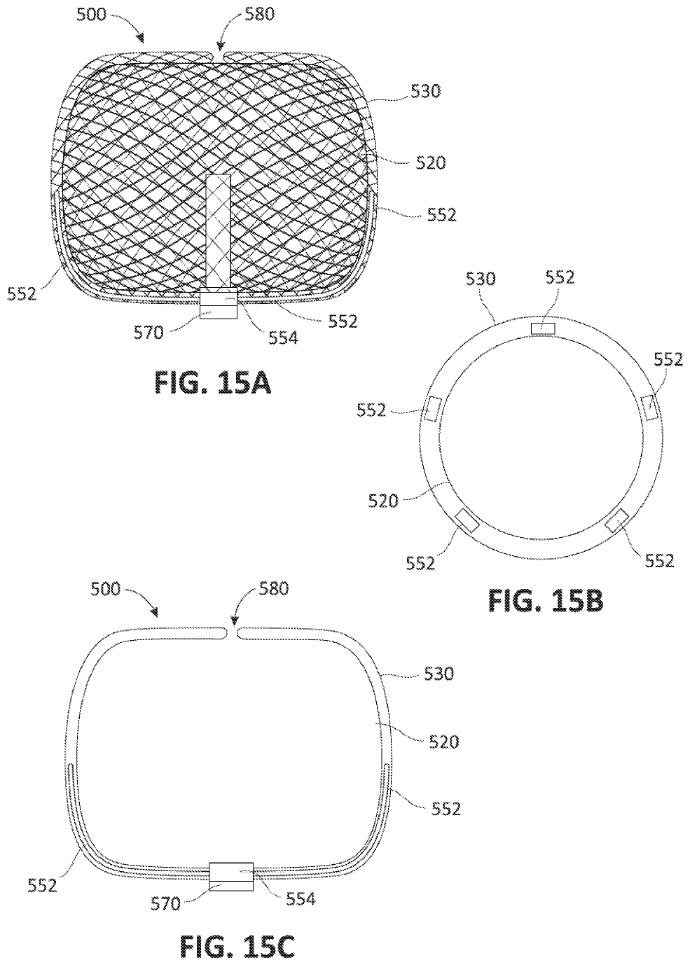

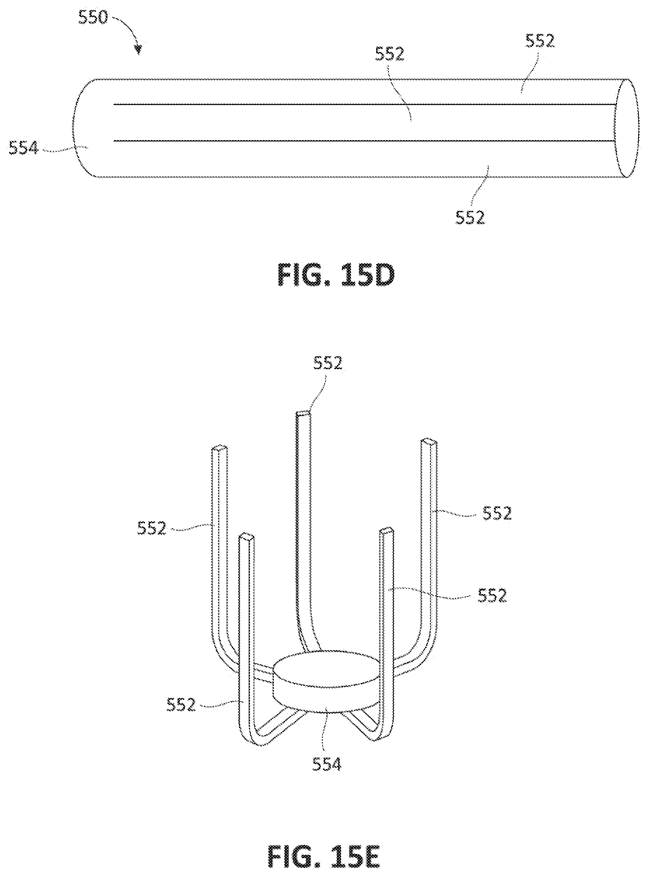

In another embodiment, an implant may include an expanding structure with a plurality of curved extensions to assist in the deployment of the implant. As seen in FIGS. 15A-15E, occlusion implant 500 can include first 520 and second 530 mesh layers formed by folding over a mesh tube as described previously. The first and second ends of each of the plurality of filaments that form the first 520 and second 530 mesh layers may be gathered in a proximal hub 570, which may also be a radiopaque marker. The distal end of implant 500 may have an opening 580 and may not include a distal marker band. The soft, atraumatic distal end may easily conform to the anatomy of the aneurysm in which it is implanted. Additional radial stiffness may be added to implant 500 by including a reinforcing structure 550 that extends from a ring 554 located at the proximal end of device 500 and extends outwardly and upwardly to a middle region of implant 500 to assist in deployment of implant 500 to its expanded state.

As seen in FIGS. 15D-15E, a structure 550 may be laser cut and heat shaped from a section of tubing. As seen in FIG. 15D, the tubing may be cut lengthwise from near one end of the tube (leaving a small portion of the end of the tube intact to form a ring 552) to the second end of the tube. The cut “fingers” or extensions 552 may be formed and heat set such that the fingers extend outwardly and upward from a ring 552 to follow the contours of the implant 500 in its expanded state, which may be substantially globular, spherical, cylindrical, or similar to a pumpkin, as described previously. As seen in FIGS. 15A-15B, the fingers 552 may extend from the ring 554 and expand in between the first 520 and second 530 mesh layers to assist the implant 500 in expanding. The fingers 552 may be a plurality of elongate extensions that extend to a middle region of the expanded state of implant 500. The fingers 552 may extend to a region located between about 25%-75%, alternatively between about 40%-60%, alternatively between about 45%-55%, alternatively about 50% of the total height of expanded state of the implant 500, from the proximal end of the implant. The fingers may include at least about 3 fingers, alternatively at least about 4 fingers, alternatively at least about 5 fingers, alternatively at least about 6 fingers, alternatively between about 3 and about 8 fingers, alternatively between about 4 and about 6 fingers.

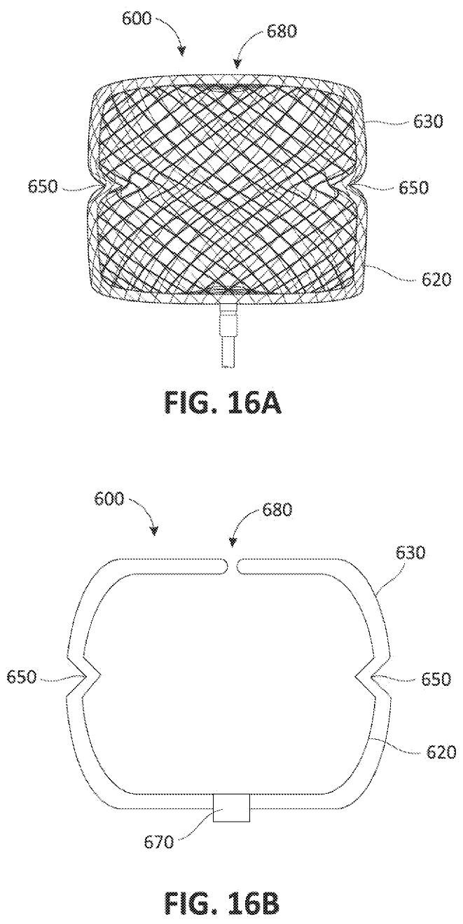

In another embodiment, an implant may include a corrugation or indentation to assist in the deployment of the implant. As seen in FIGS. 16A and 16B, occlusion implant 600 may include first 620 and second 630 mesh layers formed by folding over a mesh tube as described previously. The first and second ends of each of the plurality of filaments that form the first 620 and second 630 mesh layers may be gathered in a proximal hub 670, which may also be a radiopaque marker. The distal end of implant 600 may have an opening 680 and may not include a distal marker band. The soft, atraumatic distal end may easily conform to the anatomy of the aneurysm in which it is implanted.

Additional radial stiffness may be added to implant 600 by including a corrugation or ridge (or indentation) 650 in a middle or central region (between the distal and proximal ends of implant 600) to increase the structural stiffness of implant 600. As with corrugated metal, these extra folds would increase the strength of the overall structure and minimizing the tendency of the device to twist and fold on itself during deployment. The corrugation or ridge 650 may extend around a perimeter of the implant 600 and may be present in both the first 620 and second 630 mesh layers. The corrugation 650 may be in a region located between about 25%-75%, alternatively between about 40%-60%, alternatively between about 45%-55%, alternatively about 50% of the total height of expanded state of the implant 500, from the proximal end of the implant.

The dual layer implant 600 can be formed according to the same methods described with respect to the other implants with fold-over layers. The corrugation or ridge 650 may be added by putting a wire around a middle region of the implant 600, tightening it to draw in the implant 600, and heat setting the corrugation or ridge 650 into implant 600.

Alternatively, two smaller molds, which may be approximately the same size) may be placed inside the folded-over mesh tube and a wire may be wrapped around the outside of the folded over mesh tube in the gap between the two molds, and the implant could be heat set to form the corrugation or ridge 650. Alternatively, a single mold may have a gap in a middle region in which a wire can be wrapped around the outside of the folded over mesh tube in the gap in the single mold, and the implant could be heat set to form the corrugation or ridge 650.

All of the embodiments of permeable shells described herein have a radially constrained elongated state configured for delivery within a microcatheter, and an expanded relaxed state with a globular or barrel-like, longitudinally shortened configuration relative to the radially constrained state. In the expanded state, the permeable shell can have a maximum diameter of about 4 mm, alternatively about 5 mm, alternatively about 6 mm, alternatively about 7 mm, alternatively about 8 mm, alternatively about 9 mm, alternatively about 10 mm, alternatively about 11 mm. The expanded state of the permeable shell can have a height or length of about 2.6 mm, about 3 mm, about 3.6 mm, about 4 mm, about 4.6 mm, about 5 mm, about 5.6 mm, about 6 mm, about 6.6 mm, about 7 mm, about 7.6 mm, about 8 mm, about 8.6 mm, about 9 mm, about 9.6 mm, or about 10 mm. The woven structure of the filaments forming the mesh portions includes a plurality of openings in the permeable shell formed between the woven filaments. In some embodiments, the occlusive device may be configured as an intrasaccular occlusive device which generally conforms to the shape of the treatment site. The permeable shells may have an expanded shape that is substantially globular, spherical, barrel, cylindrical, or shaped similar to a pumpkin.

As described in more detail below, the permeable shell may be delivered by advancing a pusher detachably coupled to a proximal end of the permeable shell through a lumen of the microcatheter. Once it has been deployed in the region of interest (inner cavity of the aneurysm), the permeable shell can be detached from the pusher. The implant may be detachably coupled to the distal end of the pusher through a thermal, mechanical, or electrolytic mechanism.

For some embodiments, the permeable shell or portions thereof may be porous and may be highly permeable to liquids. In contrast to most vascular prosthesis fabrics or grafts which typically have a water permeability below 2,000 ml/min/cm² when measured at a pressure of 120 mmHg, the permeable shell 40 discussed herein may have a water permeability greater than about 2,000 ml/min/cm², in some cases greater than about 2,500 ml/min/cm². For some embodiments, water permeability of the permeable shell 40 or portions thereof may be between about 2,000 and 10,000 ml/min/cm², more specifically, about 2,000 ml/min/cm² to about 15,000 ml/min/cm², when measured at a pressure of 120 mmHg.

Device embodiments and components thereof may include metals, polymers, biologic materials and composites thereof. Suitable metals include zirconium-based alloys, cobalt-chrome alloys, nickel-titanium alloys, platinum, tantalum, stainless steel, titanium, gold, and tungsten. Potentially suitable polymers include but are not limited to acrylics, silk, silicones, polyvinyl alcohol, polypropylene, polyvinyl alcohol, polyesters (e.g. polyethylene terephthalate or PET), PolyEtherEther Ketone (PEEK), polytetrafluoroethylene (PTFE), polycarbonate urethane (PCU) and polyurethane (PU). Device embodiments may include a material that degrades or is absorbed or eroded by the body. A bioresorbable (e.g., breaks down and is absorbed by a cell, tissue, or other mechanism within the body) or bioabsorbable (similar to bioresorbable) material may be used. Alternatively, a bioerodable (e.g., erodes or degrades over time by contact with surrounding tissue fluids, through cellular activity or other physiological degradation mechanisms), biodegradable (e.g., degrades over time by enzymatic or hydrolytic action, or other mechanism in the body), or dissolvable material may be employed. Each of these terms is interpreted to be interchangeable. bioabsorbable polymer. Potentially suitable bioabsorbable materials include polylactic acid (PLA), poly(alpha-hydroxy acid) such as poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester, poly(amino acids), or related copolymer materials. An absorbable composite fiber may be made by combining a reinforcement fiber made from a copolymer of about 18% glycolic acid and about 82% lactic acid with a matrix material consisting of a blend of the above copolymer with about 20% polycaprolactone (PCL).

Permeable shell embodiments or portions thereof may be formed at least in part of wire, ribbon, or other filamentary elements 14. These filamentary elements 14 may have circular, elliptical, ovoid, square, rectangular, or triangular cross-sections. Permeable shell embodiments 40 or portions thereof may also be formed using conventional machining, laser cutting, electrical discharge machining (EDM) or photochemical machining (PCM). If made of a metal, it may be formed from either metallic tubes or sheet material.

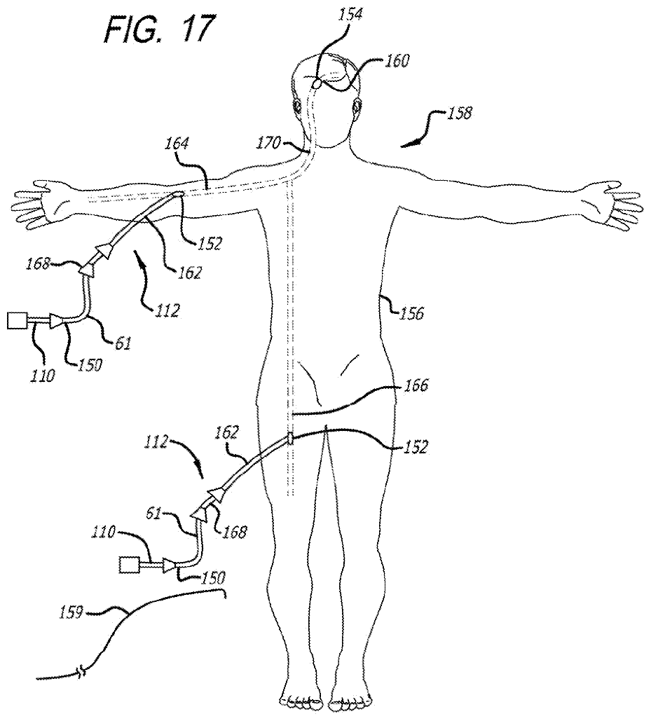

Device embodiments discussed herein may be delivered and deployed from a delivery and positioning system 112 that includes a microcatheter 61, such as the type of microcatheter 61 that is known in the art of neurovascular navigation and therapy. Device embodiments for treatment of a patient's vasculature may be elastically collapsed and restrained by a tube or other radial restraint, such as an inner lumen 120 of a microcatheter 61, for delivery and deployment. The microcatheter 61 may generally be inserted through a small incision 152 accessing a peripheral blood vessel such as the femoral artery or brachial artery. The microcatheter 61 may be delivered or otherwise navigated to a desired treatment site 154 from a position outside the patient's body 156 over a guidewire 159 under fluoroscopy or by other suitable guiding methods. The guidewire 159 may be removed during such a procedure to allow insertion of the device 10, 210 secured to a delivery apparatus 111 of the delivery system 112 through the inner lumen 120 of a microcatheter 61 in some cases. FIG. 17 illustrates a schematic view of a patient 158 undergoing treatment of a vascular defect 160 as shown in FIG. 18. An access sheath 162 is shown disposed within either a radial artery 164 or femoral artery 166 of the patient 158 with a delivery system 112 that includes a microcatheter 61 and delivery apparatus 111 disposed within the access sheath 162. The delivery system 112 is shown extending distally into the vasculature of the patient's brain adjacent a vascular defect 160 in the patient's brain.

Access to a variety of blood vessels of a patient may be established, including arteries such as the femoral artery 166, radial artery 164, and the like in order to achieve percutaneous access to a vascular defect 160. In general, the patient 158 may be prepared for surgery and the access artery is exposed via a small surgical incision 152 and access to the lumen is gained using the Seldinger technique where an introducing needle is used to place a wire over which a dilator or series of dilators dilates a vessel allowing an introducer sheath 162 to be inserted into the vessel. This would allow the device to be used percutaneously. With an introducer sheath 162 in place, a guiding catheter 168 is then used to provide a safe passageway from the entry site to a region near the target site 154 to be treated. For example, in treating a site in the human brain, a guiding catheter 168 would be chosen which would extend from the entry site 152 at the femoral artery up through the large arteries extending around the heart through the aortic arch, and downstream through one of the arteries extending from the upper side of the aorta such as the carotid artery 170. Typically, a guidewire 159 and neurovascular microcatheter 61 are then placed through the guiding catheter 168 and advanced through the patient's vasculature, until a distal end 151 of the microcatheter 61 is disposed adjacent or within the target vascular defect 160, such as an aneurysm. Exemplary guidewires 159 for neurovascular use include the Synchro2® made by Boston Scientific and the Glidewire Gold Neuro® made by MicroVention Terumo. Typical guidewire sizes may include 0.014 inches and 0.018 inches. Once the distal end 151 of the catheter 61 is positioned at the site, often by locating its distal end through the use of radiopaque marker material and fluoroscopy, the catheter is cleared. For example, if a guidewire 159 has been used to position the microcatheter 61, it is withdrawn from the catheter 61 and then the implant delivery apparatus 111 is advanced through the microcatheter 61.

Delivery and deployment of device embodiments discussed herein may be carried out by first compressing the device to a radially constrained and longitudinally flexible state as shown in FIG. 11. The device may then be delivered to a desired treatment site 154 while disposed within the microcatheter 61, and then ejected or otherwise deployed from a distal end 151 of the microcatheter 61. In other method embodiments, the microcatheter 61 may first be navigated to a desired treatment site 154 over a guidewire 159 or by other suitable navigation techniques. The distal end of the microcatheter 61 may be positioned such that a distal port of the microcatheter 61 is directed towards or disposed within a vascular defect 160 to be treated and the guidewire 159 withdrawn. The device secured to a suitable delivery apparatus 111 may then be radially constrained, inserted into a proximal portion of the inner lumen 120 of the microcatheter 61 and distally advanced to the vascular defect 160 through the inner lumen 120.

Once disposed within the vascular defect 160, the device may then allowed to assume an expanded relaxed or partially relaxed state with the permeable shell 40 or portions thereof of the device spanning or partially spanning a portion of the vascular defect 160 or the entire vascular defect 160. The device may also be activated by the application of an energy source to assume an expanded deployed configuration once ejected from the distal section of the microcatheter 61 for some embodiments. Once the device is deployed at a desired treatment site 154, the microcatheter 61 may then be withdrawn.

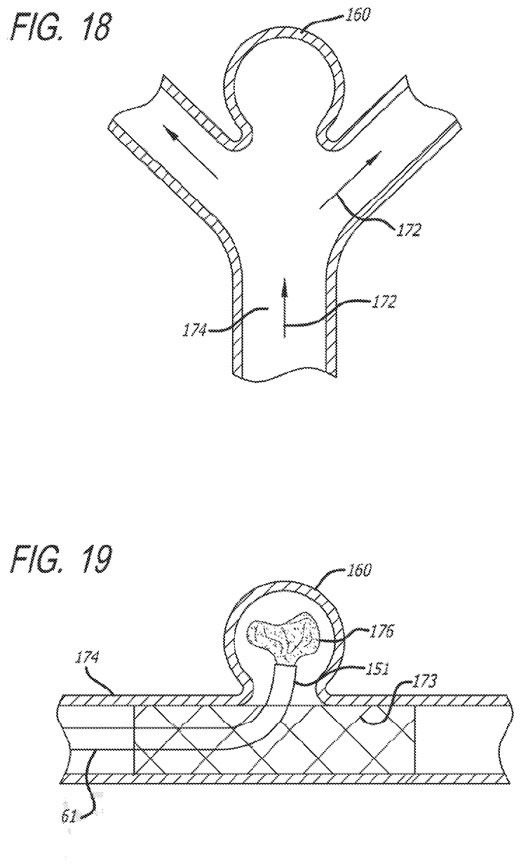

Some embodiments of devices for the treatment of a patient's vasculature discussed herein may be directed to the treatment of specific types of defects of a patient's vasculature. For example, referring to FIG. 18, an aneurysm 160 commonly referred to as a terminal aneurysm is shown in section. Terminal aneurysms occur typically at bifurcations in a patient's vasculature where blood flow, indicated by the arrows 172, from a supply vessel splits into two or more branch vessels directed away from each other. The main flow of blood from the supply vessel 174, such as a basilar artery, sometimes impinges on the vessel where the vessel diverges and where the aneurysm sack forms. Terminal aneurysms may have a well-defined neck structure where the profile of the aneurysm 160 narrows adjacent the nominal vessel profile, but other terminal aneurysm embodiments may have a less defined neck structure or no neck structure. FIG. 19 illustrates a typical berry type aneurysm 160 in section where a portion of a wall of a nominal vessel section weakens and expands into a sack like structure ballooning away from the nominal vessel surface and profile. Some berry type aneurysms may have a well-defined neck structure as shown in FIG. 19, but others may have a less defined neck structure or none at all. FIG. 19 also shows some optional procedures wherein a stent 173 or other type of support has been deployed in the parent vessel 174 adjacent the aneurysm. Also, shown is embolic material 176 being deposited into the aneurysm 160 through a microcatheter 61. Either or both of the stent 173 and embolic material 176 may be so deployed either before or after the deployment of a device for treatment of a patient's vasculature 10.

Prior to delivery and deployment of a device for treatment of a patient's vasculature, it may be desirable for the treating physician to choose an appropriately sized device to optimize the treatment results. Some embodiments of treatment may include estimating a volume of a vascular site or defect 160 to be treated and selecting a device with a volume that is substantially the same volume or slightly over-sized relative to the volume of the vascular site or defect 160. The volume of the vascular defect 160 to be occluded may be determined using three-dimensional angiography or other similar imaging techniques along with software which calculates the volume of a selected region. The amount of over-sizing may be between about 2% and 15% of the measured volume. In some embodiments, such as a very irregular shaped aneurysm, it may be desirable to under-size the volume of the device. Small lobes or “daughter aneurysms” may be excluded from the volume, defining a truncated volume which may be only partially filled by the device without affecting the outcome. A device deployed within such an irregularly shaped aneurysm 160 is shown in FIG. 28 discussed below. Such a method embodiment may also include implanting or deploying the device so that the vascular defect 160 is substantially filled volumetrically by a combination of device and blood contained therein. The device may be configured to be sufficiently conformal to adapt to irregular shaped vascular defects 160 so that at least about 75%, in some cases about 80%, of the vascular defect volume is occluded by a combination of device 10, 210 and blood contained therein.

In particular, for some treatment embodiments, it may be desirable to choose a device that is properly oversized in a transverse dimension so as to achieve a desired conformance, radial force and fit after deployment of the device. FIGS. 20-22 illustrate a schematic representation of how a device may be chosen for a proper fit after deployment that is initially oversized in a transverse dimension by at least about 10% of the largest transverse dimension of the vascular defect 160 and sometimes up to about 100% of the largest transverse dimension. For some embodiments, the device may be oversized a small amount (e.g. less than about 1.5 mm) in relation to measured dimensions for the width, height or neck diameter of the vascular defect 160.

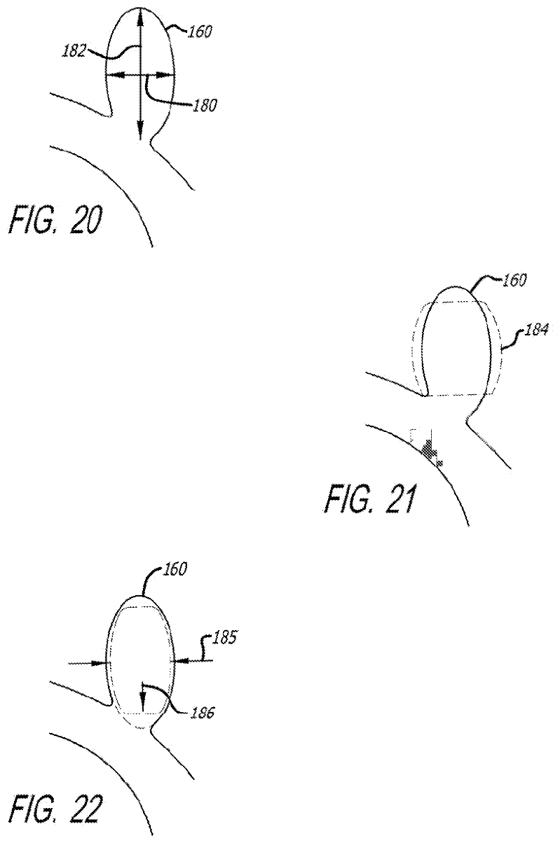

In FIG. 20, a vascular defect 160 in the form of a cerebral aneurysm is shown with horizontal arrows 180 and vertical arrows 182 indicating the approximate largest interior dimensions of the defect 160. Arrow 180 extending horizontally indicates the largest transverse dimension of the defect 160. In FIG. 21, a dashed outline 184 of a device for treatment of the vascular defect is shown superimposed over the vascular defect 160 of FIG. 20 illustrating how a device that has been chosen to be approximately 20% oversized in a transverse dimension would look in its unconstrained, relaxed state. FIG. 22 illustrates how the device, which is indicated by the dashed line 184 of FIG. 21 might conform to the interior surface of the vascular defect 160 after deployment whereby the nominal transverse dimension of the device in a relaxed unconstrained state has now been slightly constrained by the inward radial force 185 exerted by the vascular defect 160 on the device. In response, as the filaments 14, 214 of the device and thus the permeable shell made therefrom have a constant length, the device has assumed a slightly elongated shape in the axial or longitudinal axis of the device so as to elongate and better fill the interior volume of the defect 160 as indicated by the downward arrow 186 in FIG. 22.

Once a properly sized device has been selected, the delivery and deployment process may then proceed. It should also be noted also that the properties of the device embodiments and delivery system embodiments 112 discussed herein generally allow for retraction of a device after initial deployment into a defect 160, but before detachment of the device. Therefore, it may also be possible and desirable to withdraw or retrieve an initially deployed device after the fit within the defect 160 has been evaluated in favor of a differently sized device. An example of a terminal aneurysm 160 is shown in FIG. 23 in section. The tip 151 of a catheter, such as a microcatheter 61 may be advanced into or adjacent the vascular site or defect 160 (e.g. aneurysm) as shown in FIG. 24. For some embodiments, an embolic coil or other vaso-occlusive device or material 176 (as shown for example in FIG. 19) may optionally be placed within the aneurysm 160 to provide a framework for receiving the device. In addition, a stent 173 may be placed within a parent vessel 174 of some aneurysms substantially crossing the aneurysm neck prior to or during delivery of devices for treatment of a patient's vasculature discussed herein (also as shown for example in FIG. 19). An example of a suitable microcatheter 61 having an inner lumen diameter of about 0.020 inches to about 0.022 inches is the Rapid Transit® manufactured by Cordis Corporation. Examples of some suitable microcatheters 61 may include microcatheters having an inner lumen diameter of about 0.026 inch to about 0.028 inch, such as the Rebar® by Ev3 Company, the Renegade Hi-Flow® by Boston Scientific Corporation, and the Mass Transit® by Cordis Corporation. Suitable microcatheters having an inner lumen diameter of about 0.031 inch to about 0.033 inch may include the Marksmen® by Chestnut Medical Technologies, Inc. and the Vasco 28® by Balt Extrusion. A suitable microcatheter 61 having an inner lumen diameter of about 0.039 inch to about 0.041 inch includes the Vasco 35 by Balt Extrusion. These microcatheters 61 are listed as exemplary embodiments only, other suitable microcatheters may also be used with any of the embodiments discussed herein.

Detachment of the device from the delivery apparatus 111 may be controlled by a control switch 188 disposed at a proximal end of the delivery system 112, which may also be coupled to an energy source 142, which severs the tether 72 that secures the proximal hub 68 of the device to the delivery apparatus 111. While disposed within the microcatheter 61 or other suitable delivery system 112, as shown in FIG. 11, the filaments 14, 214 of the permeable shell 40, 240 may take on an elongated, non-everted configuration substantially parallel to each other and a longitudinal axis of the catheter 61. Once the device is pushed out of the distal port of the microcatheter 61, or the radial constraint is otherwise removed, the distal ends 62 of the filaments may then axially contract towards each other so as to assume the globular everted configuration within the vascular defect 160 as shown in FIG. 25.

The device may be inserted through the microcatheter 61 such that the catheter lumen 120 restrains radial expansion of the device during delivery. Once the distal tip or deployment port of the delivery system 112 is positioned in a desirable location adjacent or within a vascular defect 160, the device may be deployed out the distal end of the catheter 61 thus allowing the device to begin to radially expand as shown in FIG. 25. As the device emerges from the distal end of the delivery system 112, the device expands to an expanded state within the vascular defect 160, but may be at least partially constrained by an interior surface of the vascular defect 160.

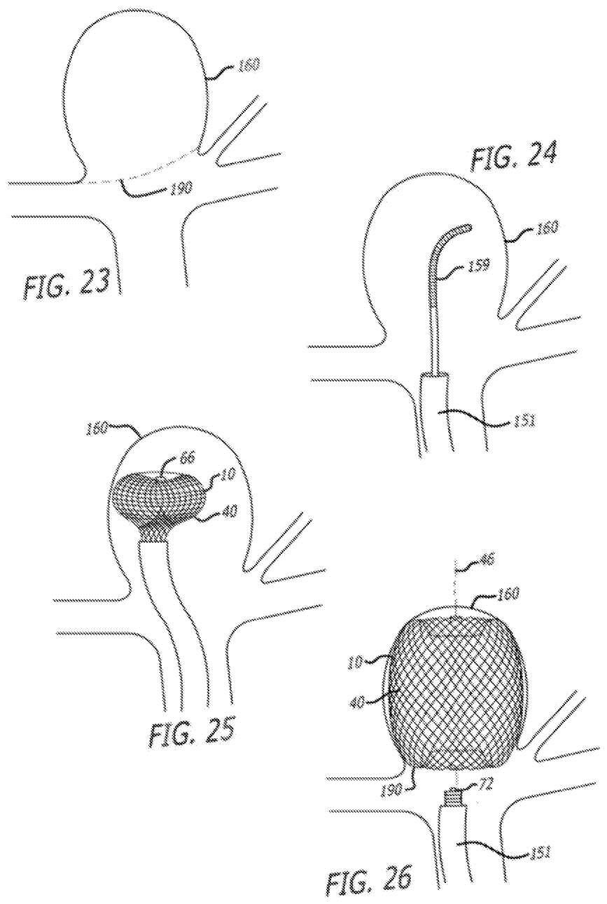

Upon full deployment, radial expansion of the device may serve to secure the device within the vascular defect 160 and also deploy the device across at least a portion of an opening 190 (e.g. aneurysm neck) so as to at least partially isolate the vascular defect 160 from flow, pressure or both of the patient's vasculature adjacent the vascular defect 160 as shown in FIG. 26. The conformability of the device, particularly in the neck region 190 may provide for improved sealing. For some embodiments, once deployed, the device may substantially slow the flow of fluids and impede flow into the vascular site and thus reduce pressure within the vascular defect 160. For some embodiments, the device may be implanted substantially within the vascular defect 160, however, in some embodiments, a portion of the device may extend into the defect opening or neck 190 or into branch vessels.

For some embodiments, as discussed above, the device may be manipulated by the user to position the device within the vascular site or defect 160 during or after deployment but prior to detachment. For some embodiments, the device may be rotated in order to achieve a desired position of the device and, more specifically, a desired position of the permeable shell or portions thereof, prior to or during deployment of the device. For some embodiments, the device may be rotated about a longitudinal axis of the delivery system 112 with or without the transmission or manifestation of torque being exhibited along a middle portion of a delivery catheter being used for the delivery. It may be desirable in some circumstances to determine whether acute occlusion of the vascular defect 160 has occurred prior to detachment of the device from the delivery apparatus 111 of the delivery system 112. These delivery and deployment methods may be used for deployment within berry aneurysms, terminal aneurysms, or any other suitable vascular defect embodiments 160. Some method embodiments include deploying the device at a confluence of three vessels of the patient's vasculature that form a bifurcation such that the permeable shell 40 or portions thereof of the device substantially covers the neck of a terminal aneurysm. Once the physician is satisfied with the deployment, size and position of the device, the device may then be detached by actuation of the control switch 188 by the methods described above and shown in FIG. 26. Thereafter, the device is in an implanted state within the vascular defect 160 to effect treatment thereof.

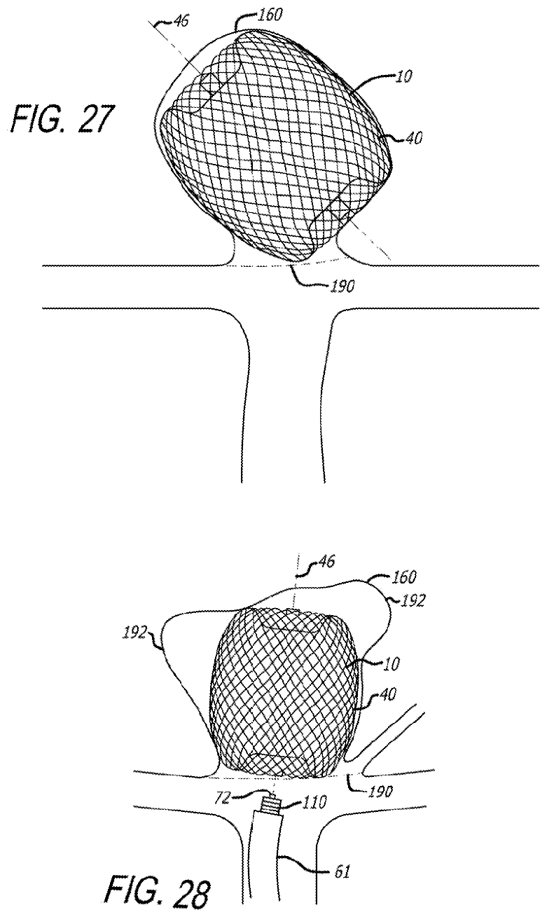

FIG. 27 illustrates another configuration of a deployed and implanted device in a patient's vascular defect 160. While the implantation configuration shown in FIG. 26 indicates a configuration whereby the longitudinal axis 46 of the device is substantially aligned with a longitudinal axis of the defect 160, other suitable and clinically effective implantation embodiments may be used. For example, FIG. 27 shows an implantation embodiment whereby the longitudinal axis 46 of the implanted device is canted at an angle of about 10 degrees to about 90 degrees relative to a longitudinal axis of the target vascular defect 160. Such an alternative implantation configuration may also be useful in achieving a desired clinical outcome with acute occlusion of the vascular defect 160 in some cases and restoration of normal blood flow adjacent the treated vascular defect. FIG. 28 illustrates a device implanted in an irregularly shaped vascular defect 160. The aneurysm 160 shown has at least two distinct lobes 192 extending from the main aneurysm cavity. The two lobes 192 shown are unfilled by the deployed vascular device, yet the lobes 192 are still isolated from the parent vessel of the patient's body due to the occlusion of the aneurysm neck portion 190.

Markers, such as radiopaque markers, on the device or delivery system 112 may be used in conjunction with external imaging equipment (e.g., x-ray) to facilitate positioning of the device or delivery system during deployment. Once the device is properly positioned, the device may be detached by the user. For some embodiments, the detachment of the device from the delivery apparatus 111 of the delivery system 112 may be affected by the delivery of energy (e.g., heat, radiofrequency, ultrasound, vibrational, or laser) to a junction or release mechanism between the device and the delivery apparatus 111. Once the device has been detached, the delivery system 112 may be withdrawn from the patient's vasculature or patient's body 158. For some embodiments, a stent 173 may be place within the parent vessel substantially crossing the aneurysm neck 190 after delivery of the device 10 as shown in FIG. 19 for illustration.

For some embodiments, a biologically active agent or a passive therapeutic agent may be released from a responsive material component of the device. The agent release may be affected by one or more of the body's environmental parameters or energy may be delivered (from an internal or external source) to the device. Hemostasis may occur within the vascular defect 160 as a result of the isolation of the vascular defect 160, ultimately leading to clotting and substantial occlusion of the vascular defect 160 by a combination of thrombotic material and the device. For some embodiments, thrombosis within the vascular defect 160 may be facilitated by agents released from the device and/or drugs or other therapeutic agents delivered to the patient.

For some embodiments, once the device has been deployed, the attachment of platelets to the permeable shell or portions thereof may be inhibited and the formation of clot within an interior space of the vascular defect 160, device, or both promoted or otherwise facilitated with a suitable choice of thrombogenic coatings, anti-thrombogenic coatings or any other suitable coatings (not shown) which may be disposed on any portion of the device for some embodiments, including an outer surface of the filaments or the proximal and distal hubs. Such a coating or coatings may be applied to any suitable portion of the permeable shell 40. Energy forms may also be applied through the delivery apparatus 111 and/or a separate catheter to facilitate fixation and/or healing of the device adjacent the vascular defect 160 for some embodiments. One or more embolic devices or embolic material 176 may also optionally be delivered into the vascular defect 160 adjacent permeable shell portion that spans the neck or opening 190 of the vascular defect 160 after the device has been deployed. For some embodiments, a stent or stent-like support device 173 may be implanted or deployed in a parent vessel adjacent the defect 160 such that it spans across the vascular defect 160 prior to or after deployment of the vascular defect treatment device.

In any of the above embodiments, the device may have sufficient radial compliance so as to be readily retrievable or retractable into a typical microcatheter 61. The proximal portion of the device, or the device as a whole for some embodiments, may be engineered or modified by the use of reduced diameter filaments, tapered filaments, or filaments oriented for radial flexure so that the device is retractable into a tube that has an internal diameter that is less than about 0.7 mm, using a retraction force less than about 2.7 Newtons (0.6 lbf) force. The force for retrieving the device into a microcatheter 61 may be between about 0.8 Newtons (0.18 lbf) and about 2.25 Newtons (0.5 lbf).

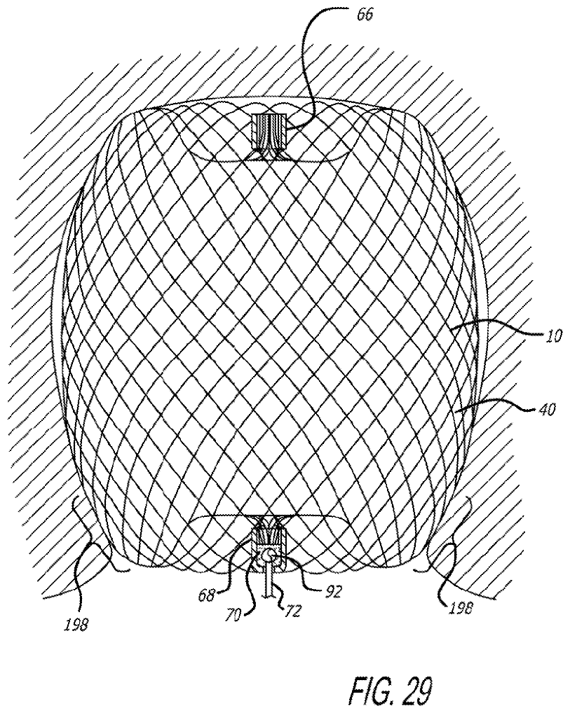

Engagement of the permeable shell 40 or portions thereof with tissue of an inner surface of a vascular defect 160, when in an expanded relaxed state, may be achieved by the exertion of an outward radial force against tissue of the inside surface of the cavity of the patient's vascular defect 160, as shown for example in FIG. 29. A similar outward radial force may also be applied by a proximal end portion and permeable shell 40 or portions thereof of the device so as to engage the permeable shell 40 with an inside surface or adjacent tissue of the vascular defect 160. Such forces may be exerted in some embodiments wherein the nominal outer transverse dimension or diameter of the device in the relaxed unconstrained state is larger than the nominal inner transverse dimension of the vascular defect 160 within which the device is being deployed, i.e., oversizing as discussed above. The elastic resiliency of the device 10, thereof may be achieved by an appropriate selection of materials, such as superelastic alloys, including nickel titanium alloys, or any other suitable material for some embodiments. The conformability of a proximal portion of the permeable shell 40 or portions thereof of the device may be such that it will readily ovalize to adapt to the shape and size of an aneurysm neck 190, as shown in FIGS. 20-22, thus providing a good seal and barrier to flow around the device. Thus, the device may achieve a good seal, substantially preventing flow around the device without the need for fixation members that protrude into the parent vessel.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many embodiments, a device for treatment of a patient's cerebral aneurysm includes a permeable shell comprising first, second, and third layers, a proximal end, a distal end, wherein the permeable shell has a radially constrained elongated state configured for delivery within a catheter lumen and an expanded state with a longitudinally shortened configuration relative to the radially constrained state, wherein the first and second layers are formed from a first plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell, and wherein the third layer is formed from a second plurality of elongate filaments that are woven together to form a mesh, wherein a first end of each of the second plurality of elongate filaments are gathered at the proximal end of the permeable shell.

In some embodiments, the mesh formed by the first plurality of elongate filaments is inverted in the middle portion to form the first and second layers.

In some embodiments, a second end of each of the second plurality of elongate filaments is gathered at the distal end of the permeable shell.

In some embodiments, each of the second plurality of elongate filaments forms a loop at or near a distal end of the permeable shell. In some embodiments, each of the first and second ends of the second plurality of elongate filaments is gathered at the proximal end of the permeable shell.

In some embodiments, the third layer is located between the first and second layers.

In some embodiments, the third layer is an innermost surface of the permeable shell.

In some embodiments, the third layer is an outermost surface of the permeable shell.

In some embodiments, the third layer is a stasis layer. In some embodiments, the first and second layers are structural layers.

In some embodiments, the first and second layers are stasis layers. In some embodiments, the third layer is a structural layer.

In some embodiments, each of the first plurality of filaments has a diameter between about 0.001″ to about 0.004″. In some embodiments, each of the second plurality of filaments has a diameter between about 0.00025″ to about 0.00075″.

In some embodiments, each of the second plurality of filaments has a diameter between about 0.001″ to about 0.004″. In some embodiments, each of the first plurality of filaments has a diameter between about 0.00025″ to about 0.00075″.

In some embodiments, an average diameter of the first plurality of filaments is larger than an average diameter of the second plurality of filaments.

In some embodiments, an average diameter of the second plurality of filaments is larger than an average diameter of the first plurality of filaments.

In some embodiments, the first plurality of filaments has a larger number of filaments than the second plurality of filaments.

In some embodiments, the second plurality of filaments has a larger number of filaments than the first plurality of filaments.

In some embodiments, the device further includes a marker band located at the proximal end of the permeable shell.

In some embodiments, the distal end of the permeable shell does not include a distal hub.

In some embodiments, the distal end of the permeable shell comprises an opening in communication with an inner cavity of the permeable shell.

In many embodiments, a method for treating a cerebral aneurysm having an interior cavity and a neck includes the steps of: advancing an implant in a microcatheter to a region of interest in a cerebral artery, wherein the implant comprises: a permeable shell comprising first, second, and third layers, a proximal end, a distal end, wherein the permeable shell has a radially constrained elongated state configured for delivery within a catheter lumen and an expanded state with a longitudinally shortened configuration relative to the radially constrained state, wherein the first and second layers are formed from a first plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell, and wherein the third layer is formed from a second plurality of elongate filaments that are woven together to form a mesh, wherein a first end of each of the second plurality of elongate filaments are gathered at the proximal end of the permeable shell; deploying the implant within the cerebral aneurysm, wherein the permeable shell expands to the expanded state in the interior cavity of the aneurysm; and withdrawing the microcatheter from the region of interest after deploying the implant.

In some embodiments, the mesh formed by the first plurality of elongate filaments is inverted in the middle portion to form the first and second layers.

In some embodiments, a second end of each of the second plurality of elongate filaments is gathered at the distal end of the permeable shell.

In some embodiments, each of the second plurality of elongate filaments forms a loop at or near a distal end of the permeable shell. In some embodiments, each of the first and second ends of the second plurality of elongate filaments is gathered at the proximal end of the permeable shell.

In some embodiments, the third layer is located between the first and second layers.

In some embodiments, the third layer is an innermost surface of the permeable shell.

In some embodiments, the third layer is an outermost surface of the permeable shell.

In some embodiments, the third layer is a stasis layer. In some embodiments, the first and second layers are structural layers.

In some embodiments, the first and second layers are stasis layers. In some embodiments, the third layer is a structural layer.

In some embodiments, each of the first plurality of filaments has a diameter between about 0.001″ to about 0.004″. In some embodiments, each of the second plurality of filaments has a diameter between about 0.00025″ to about 0.00075″.

In some embodiments, each of the second plurality of filaments has a diameter between about 0.001″ to about 0.004″. In some embodiments, each of the first plurality of filaments has a diameter between about 0.00025″ to about 0.00075″.

In some embodiments, an average diameter of the first plurality of filaments is larger than an average diameter of the second plurality of filaments.

In some embodiments, an average diameter of the second plurality of filaments is larger than an average diameter of the first plurality of filaments.

In some embodiments, the first plurality of filaments has a larger number of filaments than the second plurality of filaments.

In some embodiments, the second plurality of filaments has a larger number of filaments than the first plurality of filaments.

In some embodiments, the implant further comprises a marker band located at the proximal end of the permeable shell.

In some embodiments, the distal end of the permeable shell does not include a distal hub.

In some embodiments, the distal end of the permeable shell comprises an opening in communication with an inner cavity of the permeable shell.

In many embodiments, a device for treatment of a patient's cerebral aneurysm includes a permeable shell comprising first and second layers, a proximal end, and a distal end, and a radially expanding member located between the first and second layers, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell.

In some embodiments, the mesh formed by the plurality of elongate filaments is inverted in the middle portion to form the first and second layers.

In some embodiments, the distal end of the permeable shell does not include a distal hub.

In some embodiments, the distal end of the permeable shell comprises an opening in communication with an inner cavity of the permeable shell.

In some embodiments, the radially expanding member comprises at least one metal filament having a radially constrained elongated state configured for delivery within a catheter lumen and an expanded state, wherein in the expanded state, the at least one metal filament forms a shape comprising a loop. In some embodiments, the loop extends around a center region of the expanded state of the permeable shell.

In some embodiments, the expanded state of the at least one metal filament comprises a loop portion and a non-loop portion, wherein a proximal end of the non-loop portion is gathered at the proximal end of the permeable shell.

In many embodiments, a method for treating a cerebral aneurysm having an interior cavity and a neck includes the steps of advancing an implant in a microcatheter to a region of interest in a cerebral artery, wherein the implant comprises: a permeable shell comprising first and second layers, a proximal end, and a distal end, and a radially expanding member located between the first and second layers, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell; deploying the implant within the cerebral aneurysm, wherein the permeable shell expands to the expanded state in the interior cavity of the aneurysm; and withdrawing the microcatheter from the region of interest after deploying the implant.

In some embodiments, the mesh formed by the plurality of elongate filaments is inverted in the middle portion to form the first and second layers.

In some embodiments, the distal end of the permeable shell does not include a distal hub.

In some embodiments, the distal end of the permeable shell comprises an opening in communication with an inner cavity of the permeable shell.

In some embodiments, the radially expanding member comprises at least one metal filament having a radially constrained elongated state configured for delivery within a catheter lumen and an expanded state, wherein in the expanded state, the at least one metal filament forms a shape comprising a loop. In some embodiments, the loop extends around a center region of the expanded state of the permeable shell.

In some embodiments, the expanded state of the at least one metal filament comprises a loop portion and a non-loop portion, wherein a proximal end of the non-loop portion is gathered at the proximal end of the permeable shell.

In many embodiments, a device for treatment of a patient's cerebral aneurysm includes a permeable shell comprising first and second layers, a proximal end, and a distal end, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell; and an expandable frame comprising a core and a plurality of curved supports extending radially from the core, wherein the expandable frame has a radially constrained elongated state configured for delivery within a catheter lumen and an expanded state with a longitudinally shortened configuration relative to the radially constrained state, wherein the plurality of curved supports are located between the first and second layers of the permeable shell.

In some embodiments, the core is a ring.

In some embodiments, in the expanded state, each of the plurality of curved supports follow a contour of the expanded permeable shell.

In some embodiments, the mesh formed by the plurality of elongate filaments is inverted in the middle portion to form the first and second layers.

In some embodiments, the distal end of the permeable shell does not include a distal hub.

In some embodiments, the distal end of the permeable shell comprises an opening in communication with an inner cavity of the permeable shell.

In some embodiments, the plurality of supports comprises between about 3 and about 8 supports.

In some embodiments, the plurality of supports comprises between about 4 and about 6 supports.

In some embodiments, each of the plurality of supports comprise a proximal end and a distal end, wherein each of the plurality of supports extends towards the distal end of the permeable shell, wherein the distal ends of each of the plurality of supports are located in a region between about 40% to about 60% of the total height of expanded state of the permeable shell.

In many embodiments, a method for treating a cerebral aneurysm having an interior cavity and a neck includes the steps of advancing an implant in a microcatheter to a region of interest in a cerebral artery, wherein the implant comprises: a permeable shell comprising first and second layers, a proximal end, and a distal end, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell; and an expandable frame comprising a core and a plurality of curved supports extending radially from the core, wherein the expandable frame has a radially constrained elongated state configured for delivery within a catheter lumen and an expanded state with a longitudinally shortened configuration relative to the radially constrained state, wherein the plurality of curved supports are located between the first and second layers of the permeable shell; deploying the implant within the cerebral aneurysm, wherein the permeable shell expands to the expanded state in the interior cavity of the aneurysm; and withdrawing the microcatheter from the region of interest after deploying the implant.

In some embodiments, the core is a ring.

In some embodiments, in the expanded state, each of the plurality of curved supports follow a contour of the expanded permeable shell.

In some embodiments, the mesh formed by the plurality of elongate filaments is inverted in the middle portion to form the first and second layers.

In some embodiments, the distal end of the permeable shell does not include a distal hub.

In some embodiments, the distal end of the permeable shell comprises an opening in communication with an inner cavity of the permeable shell.

In some embodiments, the plurality of supports comprises between about 3 and about 8 supports.

In some embodiments, the plurality of supports comprises between about 4 and about 6 supports.

In some embodiments, each of the plurality of supports comprise a proximal end and a distal end, wherein each of the plurality of supports extends towards the distal end of the permeable shell, wherein the distal ends of each of the plurality of supports are located in a region between about 40% to about 60% of the total height of expanded state of the permeable shell.

In many embodiments, a device for treatment of a patient's cerebral aneurysm includes a resilient self-expanding permeable shell including a radially constrained elongated state configured for delivery within a catheter lumen, an expanded state with a longitudinally shortened configuration relative to the radially constrained state, and a plurality of elongate filaments that are woven together to form a mesh and define a cavity of the permeable shell, the expanded state having a proximal portion, a middle portion, and a distal portion, wherein the middle portion comprises a corrugation that at least partially extends around a perimeter of the permeable shell.

In some embodiments, the permeable shell comprises first and second layers. In some embodiments, the first and second layers are formed from the plurality of elongate filaments that are woven together to form the mesh, wherein each of the plurality of elongate filaments have a first end, a second end, and a middle portion, wherein the first and second ends of each of the plurality of elongate filaments are gathered at the proximal end of the permeable shell, and wherein the middle portion of each of the plurality of elongate filaments is associated with the distal end of the permeable shell. In some embodiments, both of the first and second layers of the permeable shell comprise the corrugation.

In some embodiments, the corrugation defines a plane, and wherein the plane is substantially perpendicular to an axis of the permeable shell extending from the proximal to distal end of the permeable shell.

In some embodiments, the corrugation is a ridge or indentation.

In some embodiments, the mesh formed by the plurality of elongate filaments is inverted in the middle portion to form the first and second layers.

In some embodiments, the distal end of the permeable shell does not include a distal hub.

In some embodiments, the distal end of the permeable shell comprises an opening in communication with an inner cavity of the permeable shell.

In some embodiments, the corrugation extends around the perimeter of the permeable shell.

In some embodiments, the corrugation is located in a region located about 40% to about 60% of a total height of the expanded state of the permeable shell.

In some embodiments, the distal end of the permeable shell does not include a distal marker band.

In some embodiments, the distal end of the permeable shell comprises an opening in communication with an inner cavity defined by the permeable shell.

In many embodiments, a method for treating a cerebral aneurysm having an interior cavity and a neck includes advancing an implant in a microcatheter to a region of interest in a cerebral artery, wherein the implant comprises a resilient self-expanding permeable shell including a radially constrained elongated state configured for delivery within a catheter lumen, an expanded state with a longitudinally shortened configuration relative to the radially constrained state, and a plurality of elongate filaments that are woven together to form a mesh and define a cavity of the permeable shell, the expanded state having a proximal portion, a middle portion, and a distal portion, wherein the middle portion comprises a corrugation that at least partially extends around a perimeter of the permeable shell; deploying the implant within the cerebral aneurysm, wherein the permeable shell expands to the expanded state in the interior cavity of the aneurysm; and withdrawing the microcatheter from the region of interest after deploying the implant.

In some embodiments, the permeable shell comprises first and second layers.

In some embodiments, the first and second layers are formed from the plurality of elongate filaments that are woven together to form the mesh, wherein each of the plurality of elongate filaments have a first end, a second end, and a middle portion, wherein the first and second ends of each of the plurality of elongate filaments are gathered at the proximal end of the permeable shell, and wherein the middle portion of each of the plurality of elongate filaments is associated with the distal end of the permeable shell.

Although the foregoing invention has, for the purposes of clarity and understanding, been described in some detail by way of illustration and example, it will be obvious that certain changes and modifications may be practiced which will still fall within the scope of the appended claims.

The subject matter described herein and in the accompanying figures is done so with sufficient detail and clarity to permit the inclusion of claims, at any time, in means-plus-function format pursuant to 35 U.S.C. section 112, part (f). However, a claim is to be interpreted as invoking this means-plus-function format only if the phrase “means for” is explicitly recited in that claim.

Aspects of the invention are set out in the independent claims and preferred features are set out in the dependent claims. The preferred features of the dependent claims may be provided in combination in a single embodiment and preferred features of one aspect may be provided in conjunction with other aspects.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

While the embodiments are susceptible to various modifications and alternative forms, specific examples thereof have been shown in the drawings and are herein described in detail. These embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the scope of the claims by features, functions, steps, or elements that are not within that scope.

### CLAUSES

Exemplary embodiments are set out in the following numbered clauses.

Clause 1. A device for treatment of a patient's cerebral aneurysm, comprising:

a permeable shell comprising first, second, and third layers, a proximal end, a distal end, wherein the permeable shell has a radially constrained elongated state configured for delivery within a catheter lumen and an expanded state with a longitudinally shortened configuration relative to the radially constrained state,

wherein the first and second layers are formed from a first plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell, and

wherein the third layer is formed from a second plurality of elongate filaments that are woven together to form a mesh, wherein a first end of each of the second plurality of elongate filaments are gathered at the proximal end of the permeable shell.

Clause 2. The device of clause 0, wherein the mesh formed by the first plurality of elongate filaments is inverted in the middle portion to form the first and second layers.
Clause 3. The device of clause 0, wherein a second end of each of the second plurality of elongate filaments is gathered at the distal end of the permeable shell.
Clause 4. The device of clause 0, wherein each of the second plurality of elongate filaments forms a loop at or near a distal end of the permeable shell.
Clause 5. The device of clause 0, wherein each of the first and second ends of the second plurality of elongate filaments is gathered at the proximal end of the permeable shell.
Clause 6. The device of clause 0, wherein the third layer is located between the first and second layers.
Clause 7. The device of clause 0, wherein the third layer is an innermost surface of the permeable shell.
Clause 8. The device of clause 0, wherein the third layer is an outermost surface of the permeable shell.
Clause 9. The device of clause 0, wherein the third layer is a stasis layer.
Clause 10. The device of clause 0, wherein the first and second layers are structural layers.
Clause 11. The device of clause 0, wherein the first and second layers are stasis layers.
Clause 12. The device of clause 0, wherein the third layer is a structural layer.
Clause 13. The device of clause 0, wherein each of the first plurality of filaments has a diameter between about 0.001″ to about 0.004″.
Clause 14. The device of clause 0, wherein each of the second plurality of filaments has a diameter between about 0.00025″ to about 0.00075″.
Clause 15. The device of clause 0, wherein each of the second plurality of filaments has a diameter between about 0.001″ to about 0.004″.
Clause 16. The device of clause 0, wherein each of the first plurality of filaments has a diameter between about 0.00025″ to about 0.00075″.
Clause 17. The device of clause 0, wherein an average diameter of the first plurality of filaments is larger than an average diameter of the second plurality of filaments.
Clause 18. The device of clause 0, wherein an average diameter of the second plurality of filaments is larger than an average diameter of the first plurality of filaments.
Clause 19. The device of clause 0, wherein the first plurality of filaments has a larger number of filaments than the second plurality of filaments.
Clause 20. The device of clause 0, wherein the second plurality of filaments has a larger number of filaments than the first plurality of filaments.
Clause 21. The device of clause 0, further comprising a marker band located at the proximal end of the permeable shell.
Clause 22. The device of clause 0, wherein the distal end of the permeable shell does not include a distal hub.
Clause 23. The device of clause 0, wherein the distal end of the permeable shell comprises an opening in communication with an inner cavity of the permeable shell.
Clause 24. A method for treating a cerebral aneurysm having an interior cavity and a neck, comprising the steps of:

advancing an implant in a microcatheter to a region of interest in a cerebral artery, wherein the implant comprises:
a permeable shell comprising first, second, and third layers, a proximal end, a distal end, wherein the permeable shell has a radially constrained elongated state configured for delivery within a catheter lumen and an expanded state with a longitudinally shortened configuration relative to the radially constrained state,
wherein the first and second layers are formed from a first plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell, and
wherein the third layer is formed from a second plurality of elongate filaments that are woven together to form a mesh, wherein a first end of each of the second plurality of elongate filaments are gathered at the proximal end of the permeable shell;

deploying the implant within the cerebral aneurysm, wherein the permeable shell expands to the expanded state in the interior cavity of the aneurysm; and

withdrawing the microcatheter from the region of interest after deploying the implant.

Clause 25. The method of clause 0, wherein the mesh formed by the first plurality of elongate filaments is inverted in the middle portion to form the first and second layers.
Clause 26. The method of clause 0, wherein a second end of each of the second plurality of elongate filaments is gathered at the distal end of the permeable shell.
Clause 27. The method of clause 0, wherein each of the second plurality of elongate filaments forms a loop at or near a distal end of the permeable shell.
Clause 28. The method of clause 0, wherein each of the first and second ends of the second plurality of elongate filaments is gathered at the proximal end of the permeable shell.
Clause 29. The method of clause 0, wherein the third layer is located between the first and second layers.
Clause 30. The method of clause 0, wherein the third layer is an innermost surface of the permeable shell.
Clause 31. The method of clause 0, wherein the third layer is an outermost surface of the permeable shell.
Clause 32. The method of clause 0, wherein the third layer is a stasis layer.
Clause 33. The method of clause 0, wherein the first and second layers are structural layers.
Clause 34. The method of clause 0, wherein the first and second layers are stasis layers.
Clause 35. The method of clause 0, wherein the third layer is a structural layer.
Clause 36. The method of clause 0, wherein each of the first plurality of filaments has a diameter between about 0.001″ to about 0.004″.
Clause 37. The method of clause 0, wherein each of the second plurality of filaments has a diameter between about 0.00025″ to about 0.00075″.
Clause 38. The method of clause 0, wherein each of the second plurality of filaments has a diameter between about 0.001″ to about 0.004″.
Clause 39. The method of clause 0, wherein each of the first plurality of filaments has a diameter between about 0.00025″ to about 0.00075″.
Clause 40. The method of clause 0, wherein an average diameter of the first plurality of filaments is larger than an average diameter of the second plurality of filaments.
Clause 41. The method of clause 0, wherein an average diameter of the second plurality of filaments is larger than an average diameter of the first plurality of filaments.
Clause 42. The method of clause 0, wherein the first plurality of filaments has a larger number of filaments than the second plurality of filaments.
Clause 43. The method of clause 0, wherein the second plurality of filaments has a larger number of filaments than the first plurality of filaments.
Clause 44. The method of clause 0, wherein the implant further comprises a marker band located at the proximal end of the permeable shell.
Clause 45. The method of clause 0, wherein the distal end of the permeable shell does not include a distal hub.
Clause 46. The method of clause 0, wherein the distal end of the permeable shell comprises an opening in communication with an inner cavity of the permeable shell.
Clause 47. A device for treatment of a patient's cerebral aneurysm, comprising:

a permeable shell comprising first and second layers, a proximal end, and a distal end, and a radially expanding member located between the first and second layers, wherein the permeable shell has a radially constrained elongated state configured for delivery within a catheter lumen and an expanded state with a longitudinally shortened configuration relative to the radially constrained state,

wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell.

Clause 48. The device of clause 0, wherein the mesh formed by the plurality of elongate filaments is inverted in the middle portion to form the first and second layers.
Clause 49. The device of clause 0, wherein the distal end of the permeable shell does not include a distal hub.
Clause 50. The device of clause 0, wherein the distal end of the permeable shell comprises an opening in communication with an inner cavity of the permeable shell.
Clause 51. The device of clause 0, wherein the radially expanding member comprises at least one metal filament having a radially constrained elongated state configured for delivery within a catheter lumen and an expanded state, wherein in the expanded state, the at least one metal filament forms a shape comprising a loop.
Clause 52. The device of clause 0, wherein the loop extends around a center region of the expanded state of the permeable shell.
Clause 53. The device of clause 0, wherein the expanded state of the at least one metal filament comprises a loop portion and a non-loop portion, wherein a proximal end of the non-loop portion is gathered at the proximal end of the permeable shell.
Clause 54. A method for treating a cerebral aneurysm having an interior cavity and a neck, comprising the steps of:

advancing an implant in a microcatheter to a region of interest in a cerebral artery, wherein the implant comprises:
a permeable shell comprising first and second layers, a proximal end, and a distal end, and a radially expanding member located between the first and second layers, wherein the permeable shell has a radially constrained elongated state configured for delivery within a catheter lumen and an expanded state with a longitudinally shortened configuration relative to the radially constrained state,
wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell;
   deploying the implant within the cerebral aneurysm, wherein the permeable shell expands to the expanded state in the interior cavity of the aneurysm; and

withdrawing the microcatheter from the region of interest after deploying the implant.

Clause 55. The method of clause 0, wherein the mesh formed by the plurality of elongate filaments is inverted in the middle portion to form the first and second layers.
Clause 56. The method of clause 0, wherein the distal end of the permeable shell does not include a distal hub.
Clause 57. The method of clause 0, wherein the distal end of the permeable shell comprises an opening in communication with an inner cavity of the permeable shell.
Clause 58. The method of clause 0, wherein the radially expanding member comprises at least one metal filament having a radially constrained elongated state configured for delivery within a catheter lumen and an expanded state, wherein in the expanded state, the at least one metal filament forms a shape comprising a loop.
Clause 59. The method of clause 0, wherein the loop extends around a center region of the expanded state of the permeable shell.
Clause 60. The method of clause 0, wherein the expanded state of the at least one metal filament comprises a loop portion and a non-loop portion, wherein a proximal end of the non-loop portion is gathered at the proximal end of the permeable shell.
Clause 61. A device for treatment of a patient's cerebral aneurysm, comprising:

a permeable shell comprising first and second layers, a proximal end, and a distal end, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell; and

an expandable frame comprising a core and a plurality of curved supports extending radially from the core, wherein the expandable frame has a radially constrained elongated state configured for delivery within a catheter lumen and an expanded state with a longitudinally shortened configuration relative to the radially constrained state, wherein the plurality of curved supports are located between the first and second layers of the permeable shell.

Clause 62. The device of clause 0, wherein the core is a ring.
Clause 63. The device of clause 0, wherein, in the expanded state, each of the plurality of curved supports follow a contour of the expanded permeable shell.
Clause 64. The device of clause 0, wherein the mesh formed by the plurality of elongate filaments is inverted in the middle portion to form the first and second layers.
Clause 65. The device of clause 0, wherein the distal end of the permeable shell does not include a distal hub.
66. The device of clause 0, wherein the distal end of the permeable shell comprises an opening in communication with an inner cavity of the permeable shell.
Clause 67. The device of clause 0, wherein the plurality of supports comprises between about 3 and about 8 supports.
Clause 68. The device of clause 0, wherein the plurality of supports comprises between about 4 and about 6 supports.
Clause 69. The device of clause 0, wherein each of the plurality of supports comprise a proximal end and a distal end, wherein each of the plurality of supports extends towards the distal end of the permeable shell, wherein the distal ends of each of the plurality of supports are located in a region between about 40% to about 60% of the total height of expanded state of the permeable shell.
Clause 70. A method for treating a cerebral aneurysm having an interior cavity and a neck, comprising the steps of:

advancing an implant in a microcatheter to a region of interest in a cerebral artery, wherein the implant comprises:
a permeable shell comprising first and second layers, a proximal end, and a distal end, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell; and
an expandable frame comprising a core and a plurality of curved supports extending radially from the core, wherein the expandable frame has a radially constrained elongated state configured for delivery within a catheter lumen and an expanded state with a longitudinally shortened configuration relative to the radially constrained state, wherein the plurality of curved supports are located between the first and second layers of the permeable shell;

deploying the implant within the cerebral aneurysm, wherein the permeable shell expands to the expanded state in the interior cavity of the aneurysm; and

withdrawing the microcatheter from the region of interest after deploying the implant.

Clause 71. The method of clause 0, wherein the core is a ring.
Clause 72. The method of clause 0, wherein, in the expanded state, each of the plurality of curved supports follow a contour of the expanded permeable shell.
Clause 73. The method of clause 0, wherein the mesh formed by the plurality of elongate filaments is inverted in the middle portion to form the first and second layers.
Clause 74. The method of clause 0, wherein the distal end of the permeable shell does not include a distal hub.
Clause 75. The method of clause 0, wherein the distal end of the permeable shell comprises an opening in communication with an inner cavity of the permeable shell.
Clause 76. The method of clause 0, wherein the plurality of supports comprises between about 3 and about 8 supports.
Clause 77. The method of clause 0, wherein the plurality of supports comprises between about 4 and about 6 supports.
Clause 78. The method of clause 0, wherein each of the plurality of supports comprise a proximal end and a distal end, wherein each of the plurality of supports extends towards the distal end of the permeable shell, wherein the distal ends of each of the plurality of supports are located in a region between about 40% to about 60% of the total height of expanded state of the permeable shell.
Clause 79. A device for treatment of a patient's cerebral aneurysm, comprising:

a resilient self-expanding permeable shell including a radially constrained elongated state configured for delivery within a catheter lumen, an expanded state with a longitudinally shortened configuration relative to the radially constrained state, and a plurality of elongate filaments that are woven together to form a mesh and define a cavity of the permeable shell, the expanded state having a proximal portion, a middle portion, and a distal portion, wherein the middle portion comprises a corrugation that at least partially extends around a perimeter of the permeable shell.

Clause 80. The device of clause 0, wherein the permeable shell comprises first and second layers.
Clause 81. The device of clause 0, wherein the first and second layers are formed from the plurality of elongate filaments that are woven together to form the mesh, wherein each of the plurality of elongate filaments have a first end, a second end, and a middle portion, wherein the first and second ends of each of the plurality of elongate filaments are gathered at the proximal end of the permeable shell, and wherein the middle portion of each of the plurality of elongate filaments is associated with the distal end of the permeable shell.
Clause 82. The device of clause 0, wherein both of the first and second layers of the permeable shell comprise the corrugation.
Clause 83. The device of clause 0, wherein the corrugation defines a plane, and wherein the plane is substantially perpendicular to an axis of the permeable shell extending from the proximal to distal end of the permeable shell.
Clause 84. The device of clause 0, wherein the corrugation is a ridge or indentation.
Clause 85. The device of clause 0, wherein the mesh formed by the plurality of elongate filaments is inverted in the middle portion to form the first and second layers.
Clause 86. The device of clause 0, wherein the distal end of the permeable shell does not include a distal hub.
Clause 87. The device of clause 0, wherein the distal end of the permeable shell comprises an opening in communication with an inner cavity of the permeable shell.
Clause 88. The device of clause 0, wherein the corrugation extends around the perimeter of the permeable shell.
Clause 89. The device of clause 0, wherein the corrugation is located in a region located about 40% to about 60% of a total height of the expanded state of the permeable shell.
Clause 90. The device of clause 0, wherein the distal end of the permeable shell does not include a distal marker band.
Clause 91. The device of clause 0, wherein the distal end of the permeable shell comprises an opening in communication with an inner cavity defined by the permeable shell.
Clause 92. A method for treating a cerebral aneurysm having an interior cavity and a neck, comprising the steps of:

advancing an implant in a microcatheter to a region of interest in a cerebral artery, wherein the implant comprises a resilient self-expanding permeable shell including a radially constrained elongated state configured for delivery within a catheter lumen, an expanded state with a longitudinally shortened configuration relative to the radially constrained state, and a plurality of elongate filaments that are woven together to form a mesh and define a cavity of the permeable shell, the expanded state having a proximal portion, a middle portion, and a distal portion, wherein the middle portion comprises a corrugation that at least partially extends around a perimeter of the permeable shell;

deploying the implant within the cerebral aneurysm, wherein the permeable shell expands to the expanded state in the interior cavity of the aneurysm; and

withdrawing the microcatheter from the region of interest after deploying the implant.

Clause 93. The method of clause 0, wherein the permeable shell comprises first and second layers.
Clause 94. The method of clause 0, wherein the first and second layers are formed from the plurality of elongate filaments that are woven together to form the mesh, wherein each of the plurality of elongate filaments have a first end, a second end, and a middle portion, wherein the first and second ends of each of the plurality of elongate filaments are gathered at the proximal end of the permeable shell, and wherein the middle portion of each of the plurality of elongate filaments is associated with the distal end of the permeable shell.

1. A device for treatment of a patient's cerebral aneurysm, comprising:
a permeable shell comprising first, second, and third layers, a proximal end, a distal end, wherein the permeable shell has a radially constrained elongated state configured for delivery within a catheter lumen and an expanded state with a longitudinally shortened configuration relative to the radially constrained state,
wherein the first and second layers are formed from a first plurality of elongate filaments that are woven together to form a mesh, wherein each of the first plurality of elongate filaments have a first end, a second end, and a middle portion, wherein the first and second ends of each of the first plurality of elongate filaments are gathered at the proximal end of the permeable shell, and wherein the middle portion is associated with the distal end of the permeable shell, and
wherein the third layer is formed from a second plurality of elongate filaments that are woven together to form a mesh, wherein a first end of each of the second plurality of elongate filaments are gathered at the proximal end of the permeable shell.

2. The device of claim 1, wherein the mesh formed by the first plurality of elongate filaments is inverted in the middle portion to form the first and second layers.

3. The device of claim 1, wherein a second end of each of the second plurality of elongate filaments is gathered at the distal end of the permeable shell.

4. The device of claim 1, wherein each of the second plurality of elongate filaments forms a loop at or near a distal end of the permeable shell.

5. The device of claim 4, wherein each of the first and second ends of the second plurality of elongate filaments is gathered at the proximal end of the permeable shell.

6. The device of claim 1, wherein the third layer is located between the first and second layers.

7. The device of claim 1, wherein the third layer is an innermost surface of the permeable shell.

8. The device of claim 1, wherein the third layer is an outermost surface of the permeable shell.

9. The device of claim 1, wherein the third layer is a stasis layer.

10. The device of claim 9, wherein the first and second layers are structural layers.

11. The device of claim 1, wherein the first and second layers are stasis layers.

12. The device of claim 11, wherein the third layer is a structural layer.

13. The device of claim 1, wherein each of the first plurality of filaments has a diameter between about 0.001″ to about 0.004″.

14. The device of claim 13, wherein each of the second plurality of filaments has a diameter between about 0.00025″ to about 0.00075″.

15. The device of claim 1, wherein each of the second plurality of filaments has a diameter between about 0.001″ to about 0.004″.

16. The device of claim 15, wherein each of the first plurality of filaments has a diameter between about 0.00025″ to about 0.00075″.

17. The device of claim 1, wherein an average diameter of the first plurality of filaments is larger than an average diameter of the second plurality of filaments.

18. The device of claim 1, wherein an average diameter of the second plurality of filaments is larger than an average diameter of the first plurality of filaments.

19. The device of claim 1, wherein the first plurality of filaments has a larger number of filaments than the second plurality of filaments.

20. The device of claim 1, wherein the second plurality of filaments has a larger number of filaments than the first plurality of filaments.

21. The device of claim 1, further comprising a marker band located at the proximal end of the permeable shell.

22. The device of claim 1, wherein the distal end of the permeable shell does not include a distal hub.

23. The device of claim 1, wherein the distal end of the permeable shell comprises an opening in communication with an inner cavity of the permeable shell.

24-94. (canceled)
